SEQUENCE LISTING (6 sequences)

SEQ ID NO: 1 - DNA, 26 bp, Unknown
  Primer
ccccggaggccgaattctttgtgcaa

SEQ ID NO: 2 - DNA, 20 bp, Unknown
  Primer
acaagcaggattgaaggcca

SEQ ID NO: 3 - DNA, 20 bp, Unknown
  Primer
catatcaccagctcaccgtc

SEQ ID NO: 4 - DNA, 110 bp, Human papillomavirus
actacaataattcatgtataaaactaagggcgtaaccgaaatcggttgaaccgaaaccggttagtataaaagcagacattttatgcaccaaaagagaactgcaatgtttc

SEQ ID NO: 5 - DNA, 4675 bp, adeno-associated virus 2
  CDS (321)..(2183)
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgtacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctccggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccggccatggcaagccacaaggacgatgaagaaaagttttttcctcagagcggggttctcatctttgggaagcaaggctcagagaaaacaaatgtgaacattgaaaaggtcatgattacagacgaagaggaaatcggaacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacacggtcagcgtggagatcgagtgggagctgcagaaggaaaacagcaaacgctggaatcccgaaattcagtacacttccaactacaacaagtctgttaatcgtggacttaccgtggatactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 6 - protein, 621 aa, adeno-associated virus 2
MPGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

(19) United States
(12) Patent Application Publication
(10) Pub. No.: US 2008/0145339 A1
(43) Pub. Date: Jun. 19, 2008

(54) Adeno-associated virus AAV Rep78 major regulatory protein mutants thereof and uses thereof

(75) Inventor: Paul L. Hermonat, Little Rock, AR (US)

Correspondence Address: Richard C. Peet;Foley & Lardner LLP, Washington Harbour, 3000 K Street, N.W., Suite 500, Washington, DC, 20007-5143, US

(73) Assignee: University of Arkansas

(21) Appl. No.: 10/969,248

(22) Filed: Oct. 21, 2004

Related U.S. Application Data
(63) Continuation of application No. 09/693,908, filed on Oct. 23, 2000, now abandoned.
(60) Provisional application No. 60/160,608, filed on Oct. 21, 1999.

(51) Int. Cl. A61K 35/76; C12N 7/01; A61K 38/00; C12N 15/00; C12N 15/09; C12Q 1/68; A61P 31/12; A61K 31/70; C07H 21/00; C07K 14/005

(52) U.S. Cl. 424/93.2; 435/235.1; 530/350; 514/2; 536/23.4; 536/23.1; 435/320.1; 514/44; 435/455; 435/6

(57) ABSTRACT

AAV Rep78 mutants comprising a modified AAV Rep78 protein that possesses different biochemical and biological functions as compared to the wild-type AAV Rep78 protein are disclosed. Particularly, the AAV Rep78 mutants that bind to at least one of a papillomavirus DNA or an AAV DNA or an oncogene or HIV DNA differently as compared to the wild-type AAV Rep78 protein, assays to select such mutants, and pharmaceutical compositions containing the AAV Rep78 mutants are disclosed. The present invention further discloses DNA sequences encoding at least one AAV Rep78 mutant that possesses different biochemical and biological functions as compared to the wild-type AAV Rep78 protein and pharmaceutical compositions comprising the DNA sequences. The present invention additionally is directed to a method of inhibiting papillomavirus associated diseases comprising administering pharmaceutical compositions containing AAV Rep78 mutants or the DNA sequences encoding the mutants. Also discloses are DNA sequences comprising the full length AAV genome modified to where the AAV Rep78 protein is replaced with an AAV Rep mutant that binds weakly or not at all to a papillomavirus DNA or an AAV DNA or both differently as compared to the wild-type AAV Rep78 protein and its use in producing recombinant AAV at increased levels production over wild-type levels of production. Further disclosed is a method of inhibiting papillomavirus-associated diseases, cancer, and HIV-associated diseases comprising administering a pharmaceutical composition comprising a wild-type AAV Rep78 or a mutant thereof to a patient afflicted with a papillomavirus-associated disease, cancer, or HIV-associated diseases. Particularly, useful in treating papillomavirus-associated diseases are the AAV Rep78 or mutant thereof that binds to nucleotides 14-56 of p97 of HPV-16 and inhibits expression of HPV oncoproteins. An AAV Rep78 regulation element of a DNA sequence comprising about nucleotides 14-56 of the nucleotide sequence of FIG. 2 and an AAV Rep78 regulatable promoter comprising the regulation element and a promoter sequence except for the HPV-16 p97 promoter.

### BACKGROUND OF THE INVENTION

The present invention is directed to adeno-associated virus (AAV) Rep78 mutants and DNA sequences encoding these mutants that possess the capability (a) to inhibit papillomavirus (PV) and PV-associated diseases or cancer or human immunodeficiency virus (HIV) and HIV-associated diseases or (b) to enhance AAV functions and DNA sequences encoding these AAV Rep78 mutants. The present invention also is directed to AAV Rep78 mutants and DNA sequences encoding these mutants that possess the capability to inhibit PV transcriptional activity, or alternatively, possess the capability to enhance AAV functions and the DNA constructs containing these DNA sequences. The present invention is directed to a method of inhibiting PV and PV-associated diseases or cancer or HIV and HIV-associated diseases comprising administering a composition comprising the AAV Rep78 mutants or the wild-type AAV Rep78 major regulatory protein (AAV Rep78 protein) or the DNA sequences encoding these mutants or proteins to a patient having a PV-associated disease or cancer or HIV-associated diseases. The present invention further is directed to DNA constructs encoding an AAV Rep78 protein or mutant thereof, operably linked to a specific inducible promoter that is induced during PV infection or cancer or HIV infection and the DNA construct's use in inhibiting PV-associated diseases or cancer or HIV-associated diseases in a patient. The present invention also provides a method for producing increased amounts of recombinant AAV (rAAV). A specific regulation element specific for AAV Rep78 binding is disclosed.

The present invention is further directed to a method of improving the level of rAAV gene therapy and pseudo-wild-type AAV production by all forms of complementor/generator systems by substituting improved, non-inhibiting AAV Rep78 mutants in place of the wild-type AAV Rep78 protein. There are many techniques for generating rAAV particles for gene therapy. These techniques are improved by using a mutant AAV Rep78, such as AAV Rep77^{LG} and AAV Rep79^{FA} genes or proteins, and other AAV Rep78 mutants that possess comparable properties, disclosed in this application that bind weakly or not at all to DNA sequences from PV, AAV, oncogenes or HIV, particularly, HIV-1 or a combination thereof, and preferably promoter sequences from these sources. The designations of these mutants indicate that the number following “Rep” is the amino acid that is changed. The specific amino acid change is shown in the superscript where the first amino acid is the amino acid in the wild-type AAV Rep78 and the second amino acid is the amino acid that the wild-type amino acid is changed to in the AAV Rep mutant. For example, for AAV Rep77^{LG}, the amino acid 77 is leucine (L) in the wild-type AAV Rep 78 and it is changed to glutamine (G) in the AAV Rep mutant. These designations apply to AAV Rep78 mutants disclosed in the present invention. These proteins have all of the activities required for the AAV life cycle but do not have the same ability to inhibit transcription that the wild-type AAV Rep78 protein does. Thus, AAV production is increased by 100-200% when using a vector containing one of these mutants, and therefore, these mutants are useful for producing higher titers of rAAV stock.

Other AAV Rep78 mutants that are useful within the context of the present invention, are mutants that bind better than wild-type AAV Rep78 to DNA sequences from PV, AAV, oncogenes or HIV, particularly, HIV-1, or a combination thereof, and preferably promoter sequences from these sources. AAV Rep 192^{HG}, is an example of one of these mutants, that binds to AAV p5 promoter and AAV TR and inhibits HPV-16 oncogenic transformation better than wild-type AAV Rep78. AAV Rep 192^{HG} and other AAV Rep78 mutants with comparable binding affinities are useful inhibitors of HPV and AAV promoters, and therefore, useful in inhibiting PV and PV-associated diseases, as well as HIV and HIV-associated diseases. These mutants also are useful in anti-HIV protocols and in anti-cancer protocols.

Infection and DNA integration by certain HPV types are central events in the generation of cervical cancer (Durst, et al., Proc. Natl. Acad. Sci. USA 80:3812-3815 (1983), Cullen, et al. J. Virol. 65:606-612 (1991)). The most common HPV type associated with cervical cancer is HPV-16, and roughly two thirds of cervical cancers contain the DNA of this virus. The HPV-16 sequence is provided in Seedorf et al., Virology 145(1):181, (1985). AAV is another virus commonly found in the anogenital region (Blacklow, et al., Proc. Natl. Acad. Sci. USA 58:1410-1415 (1967), Bantel-Schaal, et al., Virology 134:52-63 (1984), Tobiasch, et al., J. Med. Virol. 44:215-222 (1995), Han, et al., Virus Genes 12:47-52 (1996)). AAV is described as a helper dependent human parvovirus, which requires the cell to be co-infected with an adenovirus, herpes virus or pox virus, for AAV DNA replication and virus particle formation to take place. Without these helper viruses, AAV is unable to replicate, except under special circumstances. Adenovirus is believed to be the most common helper virus in vivo. When no helper virus is present, AAV will set up a latent infection by chromosomal integration, specifically in a region on chromosome 19.

Infection by AAV, in sharp contrast to the HPV, is negatively associated with cervical cancer as demonstrated by the prevalence or titer of anti-AAV antibodies (Mayor, et al., Am. J. Obstet. Gynecol. 126:100-105 (1976), Georg-Fries, et al., Virology 134:64-71 (1984)). Bidirectional interaction has been observed between AAV and PVs. One such interaction is that PVs might serve as helpers for AAV (Walz, et al., J. Gen. Virol. 78:1441-1452 (1997)), allowing for AAV DNA replication and virion production.

In addition to its negative association with PV infections, AAV is a useful viral vector for human gene therapy. The nucleotide sequence and organization of the AAV 2 genome is known and published. (Srivastava et al., J. Virol. 45 (1) 555, (1983)). AAV latently infects cells as a natural part of its life cycle AAV will latently infect the cell by chromosomal integration, usually within a 100 base pair region on human chromosome 19. Furthermore, AAV has not been found to be the etiologic agent of any disease, and is thus accurately described as being non-pathogenic. These properties suggest that AAV is an ideal gene transmitter for human gene therapy. AAV is highly effective in transducing a variety of cell types in tissue culture including cells of epithelial, fibroblastic and hematopoietic origin.

AAV Rep78 is an AAV DNA binding transcription factor and was first identified in Mendleson et al., J. Virol. 60(3) 823 (1986), as the largest of four products encoded by the AAV rep open reading frame. AAV Rep78 is required for AAV DNA replication (Hermonat et al., J. Virol. 51: 329, (1984); Tratschin et al., J. Virol. 51: 611, (1984)), for AAV gene regulation (Labow et al., J. Virol. 60: 251, (1986); Tratschin et al., Mol. Cell. Biol. 5: 3251, (1986)), and AAV Rep78 displays a variety of biochemical activities which are necessary for these biological phenotypes (Im et al., Cell. 61: 447, (1990); Ni et al., J. Virol. 68: 1128, (1994)).

AAV Rep78 is the full length product of the rep (for replication) open reading frame. The AAV Rep78 protein is critical for the AAV life cycle. In 1984, the initial genetic analysis of AAV using a series of linker insertion-frame shift mutants was carried out (Hermonat et al., J. Virol. 51: 329, (1984)). In this study, the rep, lip, and cap mutant phenotypes were discovered and the corresponding genes within AAV were mapped. The data from this study shows that mutations at map units 11, 32, and 42 were defective for DNA replication. The positioning of these mutations map the effect to the AAV Rep78 protein, as only AAV Rep78 is affected by all three mutations. There are three other Rep proteins, Rep68, Rep52, and Rep40, which are produced from the same open reading frame (ORF), and are largely subsets of the AAV Rep78 amino acid sequence. It is unclear if these proteins have any unique biochemical properties. They may serve to augment some of AAV Rep78's functions. In any case, AAV Rep78 is all that is needed for the AAV life cycle as indicated by earlier genetic studies and by recent data in which only AAV Rep78, of the four Rep proteins, was needed for generating rAAV vectors (Holscher et al., J. Virol. 69: 6880 (1995)).

AAV Rep78 interacts with DNA. One of AAV Rep78's functions is to bind to the AAV terminal repeats (TR DNA) which functions as the origin of replication (Ryan et al., J. Virol. 70:1542 (1996); McCarty et al., J. Virol. 68:4988 (1994); McCarty (1996) et al., J. Virol. 68:4998 (1994); Snyder et al., J. Virol. 67:6096 (1993); Ashktorab et al., J. Virol. 63:3034 (1989).

In earlier studies, it has been demonstrated that AAV inhibits bovine papillomavirus type 1 (BPV-1) and HPV-16-induced oncogenic transformation (Hermonat, P. L., Virology 172:253-261 (1989); Hermonat, P. L., Cancer Research 54:2278-2281 (1994); Hermonat, et al., Gynecologic Oncology 66:487-494 (1997)). Others have also observed AAV inhibition of BPV-1 and HPV-18 (Schmitt, et al., Virology 172:73-81 (1989); Horer, et al., J. Virol. 69:5485-5496 (1995); Su, et al., British J. Can. 73:1533-1537 (1996)) and HIV-1. The effect has been mapped to the AAV encoded Rep78 protein, and this protein has been shown to inhibit expression of the PV promoter just upstream of the E6 gene (p89 of BPV-1, p97 of HPV-16, and p105 of HPV-18) (Hermonat, P. L., Cancer Research 54:2278-2281 (1994); Schmitt, et al., Virology 172:73-81 (1989); Horer, et al., J. Virol. 69:5485-5496 (1995); Hermonat, et al., Virology 247(2):240-250 (1998)). Similar to PV, HIV has been shown to be inhibited by AAV Rep78 (Antoni B A. et al., J. Virology. 65(1):396-404 (1991); Rittner K. et al. Biochemical Society Transactions. 19(4):438S (1991); Oelze I. et al., J. Virology. 68(2): 1229-33 (1994)), and DNA binding is required for this inhibition (Batchu and Hermonat, FEBS Letters, 367(3): 267-71 (1995); Kokorina NA. et al., J. Human Virology 1(7):441-50 (1998)). Rep78 binds to two DNA sequences of the HIV long terminal repeat promoter, the TAR sequences (nt +23 to +42) (Batchu and Hermonat, FEBS Letters, 367(3):267-71 (1995)) and to a region just upstream of the TATA box (nt −54 to −34).

AAV Rep78 regulates a variety of heterologous genes. C—H-ras (Katz, et al., Cancer Research 46:3023-3026 (1986); Hermonat, P. L., Cancer Research 51:3373-3377 (1991); Khleif, et al., Virology 181:738-741 (1991)), c-fos (Klein-Bauernschmitt, et al., J. Virol. 66:419-4200 (1992); Hermonat, P. L., Cancer Letters 81:129-136 (1994)), c-myc (Klein-Bauernschmitt, et al., J. Virol. 66:419-4200 (1992); Hermonat, P. L., Cancer Letters 81:129-136 (1994)), and the HIV long terminal repeat (HIV-LTR) (Rittner, et al., J. Gen. Virol. 73:2977-2981 (1992); Antoni, et al., J. Virol. 64:396-404 (1991)) are down-regulated by AAV Rep78, while the c-sis promoter is up-regulated (Wonderling, et al., J. Virol. 70:4783-4786 (1996)). Still other genes are not affected, such as the murine osteosarcoma virus long terminal repeat (MSV-LTR)(Hermonat, P. L., Cancer Research 51:3373-3377 (1991)) and the human β-actin promoter (Horer, et al., J. Virol. 69:5485-5496 (1995)). The largest of 4 products encoded by the AAV rep open reading frame (Mendleson, et al., J. Virol. 60:823-832 (1986)), AAV Rep78, is required for AAV DNA replication (Hermonat, et al., J. Virol. 51:329-333 (1984); Tratschin, et al., J. Virol. 51:611-619 (1994)) and for AAV gene regulation (Labow, et al., J. Virol. 60:251-258 (1986); Tratschin, et al., Mol. Cell. Biol. 5:3251-3260 (1986)). AAV Rep78 carries out a range of biochemical activities which are necessary for its biological phenotypes (Im, et al., Cell 61:447-57 (1990); Ni, et al., J. Virol. 68:1128-1138 (1994)), including binding to promoter DNA (McCarty, et al., J. Virol. 74:4988-4997 (1994); Batchu, et al., Cancer Letters 86:23-31 (1994); Wonderling, et al., J. Virol. 71:2528-2534 (1996)), and to a variety of cellular proteins (Hermonat, et al., Biochem. and Molec. Biol. Internat. 403:409-420 (1997)), including the transcription factors Sp1 (Hermonat, et al. Cancer Research 56:5299-5304 (1996); Pereira, et al., J. Virol. 71:1747-1756 (1997)), TBP (Hermonat, et al., Virology 245:120-127 (1998)), and itself (Weitzman, et al., J. Virol. 70:2440-2448 (1996), Hermonat, et al., FEBS Letters 401:180-184 (1997), Smith, et al., J. Virol. 71:4461-4471 (1997)).

Hermonat et al., Cancer Research 51: 3373 (1994) involved the use of the complete or partial genomes and did not study the p97 promoter in isolation as does the present invention. This is underscored by the identification of a new promoter in the E7 gene (Higgins et al., J. Gen. Virol. 73 (Pt. 8):2047, (1992)), which was included in all of the plasmids used in the Hermonat et al. study.

Although Hermonat et al., Gynecologic Oncology 66: 487 (1997) suggests that AAV Rep78 specifically inhibits the HPV-16 p97 promoter, this publication itself acknowledges its own weakness by stating that the study does not mutationally map the specific AAV rep region product which is required for the inhibition of oncogenic transformation. Further, this publication suggests that AAV Rep78 affects p97 transcription based upon results obtained with a chloramphenicol acetyltransferase (CAT) assay. This assay measures both transcription and translation as the activity measured is of the resulting protein. In fact, the ability of AAV Rep78 to affect translation, and not transcription, was emphasized in Trempe et al., J. Virol. 62(1): 68 (1988). Thus, the main effect on p97 was not definitively proven by Hermonat et al., Gynecologic Oncology (1997), thus leaving open the question as to whether the effect of AAV Rep78 was on transcription or translation. The present invention specifically shows that the AAV Rep78 clearly affects p97 transcriptional levels.

The present invention analyzes AAV Rep78 mutants and their ability to bind to DNA and discloses that the favored site for AAV Rep78's binding within the HPV-16 genome is a region within the p97 promoter of the long control region (LCR), from nt 14 to 58 using the electrophoretic mobility shift assay (EMSA). These results are surprising as the p97 target sequence contains no GAGC (or GCTC) motifs, which is the core sequence of almost all AAV Rep78 DNA recognitions. This region is important for HPV-16 as it includes functional Sp1 (Gloss, et al., J. Virol. 64:5577-5584 (1990), Hoppe-Seyler, et al., J. Gen. Virol. 74:281-286 (1993)) and E2 (Androphy, et al., Nature 325:70-73 (1987), Moskaluk, et al., Proc. Natl. Acad. Sci. USA 85:1826-1830 (1988)) protein binding motifs as well as part of the origin of replication. Furthermore, AAV Rep78 amino acid substitution mutants, at positions 77, 79 or 64-65, identified in this invention, did not recognize p97 DNA. Compared to wild-type AAV Rep78, these AAV Rep78 mutants were found to be defective for inhibition of p97 promoter activity in HeLa and T-47D nuclear extracts in vitro, in a transient CAT assay, as well as defective for full inhibition of HPV-16-directed focus formation. These data, taken together, show that the AAV Rep78-p97 promoter interaction is responsible, at least in part, for AAV Rep78-mediated inhibition of HPV-16. These data indicate that the p97 promoter represents a new prototypical DNA target type for AAV Rep78. Furthermore, AAV Rep78 mutants unable to bind DNA are also defective in their ability to fully inhibit HPV-16 (pL67R)-induced oncogenic transformation, demonstrating the importance of the AAV Rep78-p97 DNA interaction. Plasmid L67R contains the oncogene ras coding sequences just downstream of the E7 gene, replacing E1. Thus pL67R is a “super” HPV-16, containing three oncogenes (E6, E7, and ras) expressed from the p97 promoter, instead of just two. Because of its increased activity, the pL76R plasmid allows for easier study of HPV-16 gene expression.

In this patent application, the designations of the AAV Rep78 protein and mutants thereof may also be designated as simply Rep 78 or Rep77^{LG} in the figures and the text.

### SUMMARY OF THE INVENTION

The present invention is directed to AAV Rep78 mutants comprising an AAV Rep78 modified protein that possesses different biochemical and biological functions as compared to the wild-type AAV Rep78 protein. Particularly, the AAV Rep78 mutants bind to at least one of PV DNA, AAV DNA, HIV DNA or an oncogene, preferably a promoter sequence, differently as compared to the wild-type AAV Rep78 protein's binding to these DNAs. The present invention also is directed to fusion proteins containing these AAV Rep78 mutants or wild-type AAV Rep78 proteins, and pharmaceutical compositions containing the AAV Rep78 mutants or fusion proteins comprising the AAV Rep78 mutants or the wild-type AAV Rep78 protein.

The present invention additionally is directed to a method of inhibiting at least one of PV DNA, AAV DNA, HIV DNA or an oncogene, preferably a promoter, whereby PV, PV-associated diseases, cancer, HIV or HIV-associated diseases are inhibited, comprising administering pharmaceutical compositions containing AAV Rep78 mutants or fusion proteins comprising the AAV Rep78 mutants or the wild-type AAV Rep78 protein to patients in need of treatment for these viruses and viral-associated diseases.

The present invention further is directed to a DNA sequence encoding at least one AAV Rep78 mutant that possesses different biochemical and biological functions as compared to the wild-type AAV Rep78 protein, DNA sequences encoding fusion proteins comprising the AAV Rep78 mutants or the wild-type AAV Rep78 protein and pharmaceutical compositions comprising a DNA construct containing these DNA sequences encoding the AAV Rep78 mutants or the wild-type AAV Rep78 protein.

The present invention is further directed to a method of treating PV, PV-associated diseases, cancer, HIV or HIV-associated diseases comprising administering the pharmaceutical composition containing a DNA construct containing these DNA sequences encoding the AAV Rep78 mutants that possesses different biochemical and biological functions as compared to the wild-type AAV Rep78 protein or DNA sequences encoding the wild-type AAV Rep78 protein to a patient afflicted with PV-associated diseases, cancer or HIV-associated diseases.

The present invention additionally is directed to a DNA sequence comprising the full length AAV genome (FLAG) modified to replace the AAV Rep78 DNA sequence with the DNA sequence encoding an AAV Rep78 mutant that possesses different biochemical and biological functions as compared to the wild-type AAV Rep78 protein.

The present invention further is directed to a method of producing rAAV comprising transducing a susceptible mammalian cell with the DNA sequence encoding the minimum AAV genome required to complement a defective rAAV and a DNA sequence encoding the rAAV to obtain increased numbers of rAAV as compared to using AAV sequences containing a wild-type AAV Rep78 genome as a complementor.

The present invention is directed to a method of treating PV-associated diseases, cancer or HIV-associated diseases comprising administering a pharmaceutical composition comprising a AAV Rep78 protein to a patient afflicted with a PV-associated disease or cancer, wherein said AAV Rep78 protein binds to a PV DNA sequence or promoter that controls the expression of a PV oncoprotein or binds to a DNA sequence or promoter that controls the expression of an oncoprotein. Alternatively, the AAV Rep78 protein binds to DNA sequences in the HIV in the HIV genome.

The present invention further is directed to a regulatable element to which AAV Rep78 binds and controls, and a regulatable promoter containing this sequence that preferably includes nucleotides 14-56 as shown in FIG. 2

The present invention also is directed to a method of selecting an AAV Rep78 mutant that possesses different biochemical and biological functions as compared to the wild-type AAV Rep78 protein comprising contacting an AAV Rep78 mutant as described in the present application and contacting the wild-type AAV Rep78 protein with at least one DNA sequence obtained from one or more of a papillomavirus, an AAV, a HIV or an oncogene for a period of time to allow binding of the mutant and the wild-type Rep78 to the DNA; determining the binding of the mutant and the wild-type Rep78, and compare the binding and select mutants having the desired binding affinities. If numerous binding assays are performed, it may not be necessary to perform the wild-type binding assay each time a Rep78 mutant is tested as the binding of the Rep78 protein will be known.

### BRIEF DESCRIPTION OF THE DRAWINGS

FIGS. 1A-D show AAV Rep78 recognition of sequences from the HPV-16 p97 promoter using affinity chromatography and EMSA experiments identifying the region of HPV-16 to which AAV Rep78 binds. The amount of protein added is given in μg in parentheses. FIG. 1A shows the use of AAV Rep78 affinity chromatography to select ³²P-labeled Pst I, Bam HI double digested HPV-16 DNA. AAV Rep78 preferentially binds a 1.8 Kb Pst I fragment from HPV-16 (nt 7003-875). FIG. 1B shows that AAV Rep78 preferentially binds sequences from the HPV-16 LCR, nt 7841-106, compared to an equally sized fragment from the MSV-LTR by EMSA analysis. FIG. 1C shows that AAV Rep78 preferentially binds nt 14-106 compared to 7841-13 by EMSA analysis. FIG. 1D shows that AAV Rep78 preferentially binds nt 14-56 compared to 57-106 by EMSA analysis.

FIG. 2 shows the sequences of the HPV-16 p97 promoter with the important elements within the immediate p97 region as labeled boxes.

FIG. 3 shows that AAV Rep78 specifically binds the minus (−) strand of p97 using an EMSA analysis of AAV Rep78 interaction with either the + or the − strand of p97 (nt 14-106). The + and − strands were generated by asymmetric PCR amplification.

FIGS. 4A-B show AAV Rep78 mutant proteins defective in binding AAV TR and the p97 DNA substrates. FIG. 4A shows an EMSA analysis of wild-type and mutant AAV Rep78 proteins binding to AAV TR DNA. Note that Rep-64^{LH}65^{TM} does not bind the AAV TR while wild-type and Rep-77^{LG} does. FIG. 4B shows an EMSA analysis of wild-type and mutant AAV Rep78 proteins binding to p97 DNA (nt 14-106). Note that in contrast to FIG. 4A, both Rep-64^{LH}65^{TM} and Rep-77^{LG} are unable to bind p97.

FIGS. 5A-B show that AAV Rep78 mutant proteins Rep-64^{LH}65^{TM} and Rep-77^{LG} are defective for inhibiting p97 promoter activity in in vitro transcription assays. Shown are representative in vitro transcription experiments based on: FIG. 5A-HeLa cell nuclear extracts (HPV positive) or FIG. 5B—T-47D cell nuclear extracts (HPV negative). Maltose-binding protein (MBP)-AAV Rep78, Rep-64^{LH}65^{TM} and Rep-77^{LG} were added to the reaction and the generation of the p97 RNA product was determined.

FIG. 6 shows that AAV Rep78 mutant genomes FLAG-64^{LH}65^{TM} and FLAG-77^{LG} are defective for inhibiting p97 promoter activity by CAT assay. Cotransfection of d110-37 (large deletion within AAV Rep78) served as a negative control for inhibition. Cotransfection with increasing doses of pSM620 (2, 4, and 8 μgs, wild-type AAV Rep78) served as a positive control for inhibition. Mutants FLAG-64^{LH}65^{TM} and FLAG-77^{LG} were similarly cotransfected.

FIG. 7 shows that AAV Rep78 mutant genomes FLAG-64^{LH}65^{TM} and FLAG-77^{LG} are defective for inhibiting HPV-16 oncogenic transformation. Shown is a representative of three focus formation assays. C127 contact inhibited murine fibroblasts were calcium phosphate transfected with 3 μgs of pL67R plus 6 μgs of the indicated AAV plasmid. d110-37 and pSM620 are the controls, encoding a large deleted AAV Rep78 and wild-type AAV Rep78 respectively. Note that FLAG-64^{LH}65^{TM} and FLAG-77^{LG} are significantly defective in inhibiting pL67R oncogenic transformation compared to pSM620.

FIGS. 8A-B show that AAV Rep78-p97 interaction is not as strong as AAV Rep78-TR interaction. FIG. 8A shows a competitive EMSA experiment analyzing wild-type and 77^{LG} AAV Rep78 proteins binding to a ³²P-labeled TR substrate with competition from unlabeled TR and p97 DNA. Three doses of competitor DNA (0.1, 0.5, and 1 μg) were added as indicated by the triangle. Note that TR DNA is a better competitor than p97. FIG. 8B shows a competitive EMSA experiment of wild-type AAV Rep78 protein binding to a ³²P-labeled p97 substrate with competition from unlabeled TR and p97 DNA. Three doses of competitor DNA (0.1, 0.5 and 1 μg) were added as indicated by the triangle. Again, note that TR DNA is a better competitor than p97.

FIGS. 9A-B show that AAV Rep78 binds sequences of the BPV-1 long control region (LCR). FIG. 9A shows an EMSA in which AAV Rep78 binds to the BPV-1 LCR (nt 7758-7030). Note that the DNA-protein complex occurs in a dosage dependent manner with increasing addition of MBP-AAV Rep78. These LCR sequences contain two E2 motifs. FIG. 9B shows a competitive EMSA demonstrating that the AAV TR is a better competitor than the BPV-1 LCR itself. 0.1 μg of MBP-Rep were added as indicated. Four doses of synthetic competitor DNA (Durst, et al., Proc. Natl. Acad. Sci. USA 80:3812-3815 (1983), 5, 20 and 40 ng) were added as indicated by the triangle. Note that the MSV-LTR DNA is not an effective competitor compared to the BPV LCR DNA.

FIG. 10 shows the in vitro transcription showing AAV Rep78 mutant inhibition of transcription.

FIG. 11 shows that FLAG-77^{LG} replicates at higher levels and that FLAG-192^{HG} replicates at slightly depressed levels compared to wild-type.

FIG. 12 shows that Rep-192^{HG} binds AAV TR (terminal repeat) DNA in a different higher order complex and with higher affinity.

FIG. 13 shows that Rep-192^{HG} binds to the p5 promoter sequences in a higher order complex than wild-type Rep78.

FIG. 14 shows the effect of several AAV Rep78 mutants on the inhibition of HPV-16-induced oncogenic foci.

FIG. 15A-C discloses the nucleotide sequence of the AAV2 genome as disclosed in Srivastava, A. et al., J. Virol. 45:555 (1983). The nucleotide sequence encoding AAV Rep78 is nucleotides 321-2186, in which nucleotides 2184-2186 are the stop codon, taa.

FIG. 16 discloses the 621 amino acid sequence of AAV Rep78 encoded by nucleotides 321-2183 as discussed above in the description of FIG. 15.

### DETAILED DESCRIPTION OF THE PREFERRED EMBODIMENT

AAV Rep78 is responsible for all enzymatic functions of the AAV life cycle as well as for the inhibition of HPVs. The present invention is directed to AAV Rep78 mutants comprising a modified AAV Rep78 protein that possesses different biochemical and biological functions as compared to the wild-type AAV Rep78 protein. These different functions can be enhanced or defective biochemical or biological functions as compared to the wild-type AAV rep78 protein. The mutants of the present invention also are useful to map the function domains of Rep 78.

More specifically, the modified. AAV Rep78 protein or AAV Rep78 mutants bind to at least one DNA sequence obtained from a PV, an oncogene, an AAV an HIV or a combinations thereof differently than the wild-type AAV Rep78 protein binds to these DNAs. The mutants are useful for the inhibition of viruses, particularly, PVs and HIV, and therefore, these mutants will be useful in the treatment of PV-associated diseases, preferably those disease caused by a HPV or BPV, but also other PVs known to persons skilled in the art are intended to be encompassed by the present invention. The mutants also are useful for treating the diseases that result from the expression of an oncogene or HIV DNA.

The present invention is intended to encompass two different functional types of AAV Rep78 mutants. The first type of AAV Rep78 mutant has enhanced strong binding to PV DNA or oncogenes or AAV DNA or HIV DNA or a combination of one or more of these DNAs, preferably promoter regions of these DNAs, and provides enhanced control of PV, oncogene and HIV expression. The second type of AAV Rep78 mutant has weak or no binding affinity to PV or AAV DNA or both, yet has other intact AAV functions and provides for improved AAV and rAAV production, and controls PV by increasing the numbers of AAVs.

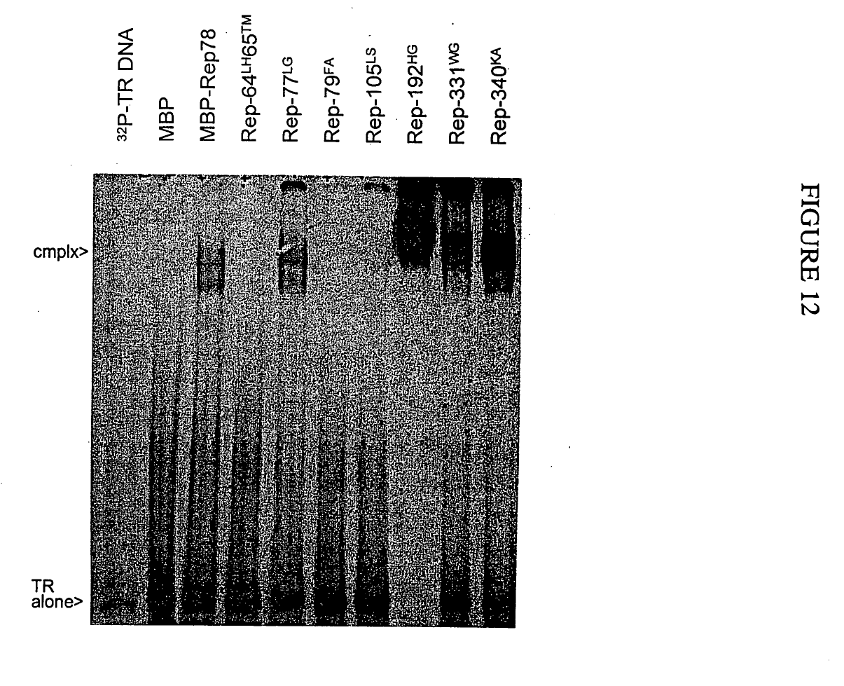

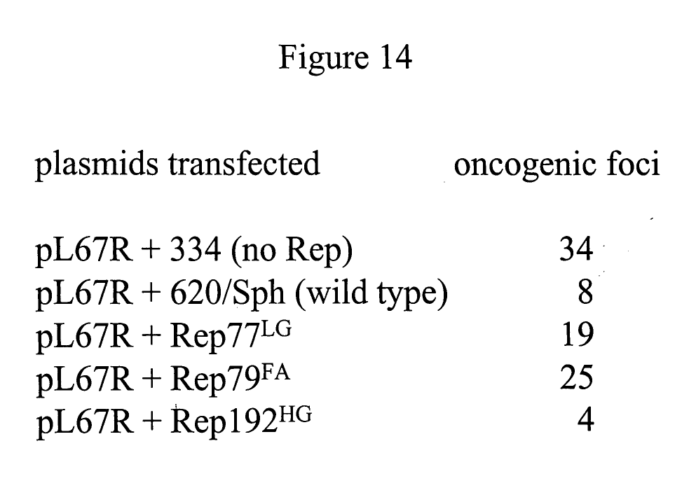

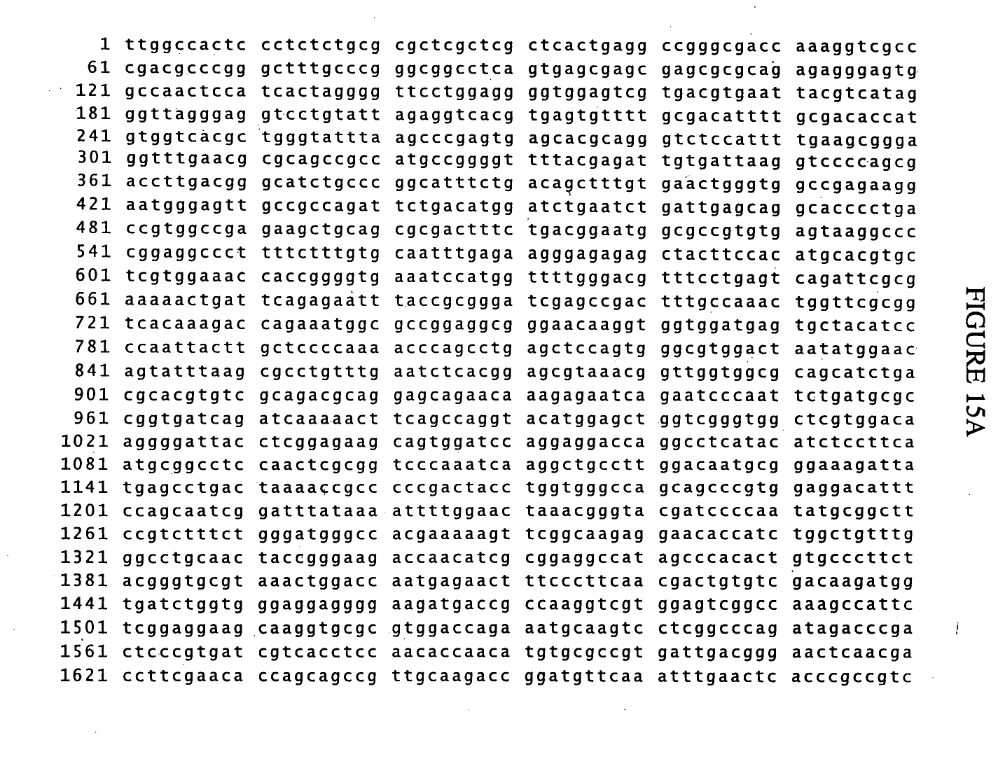

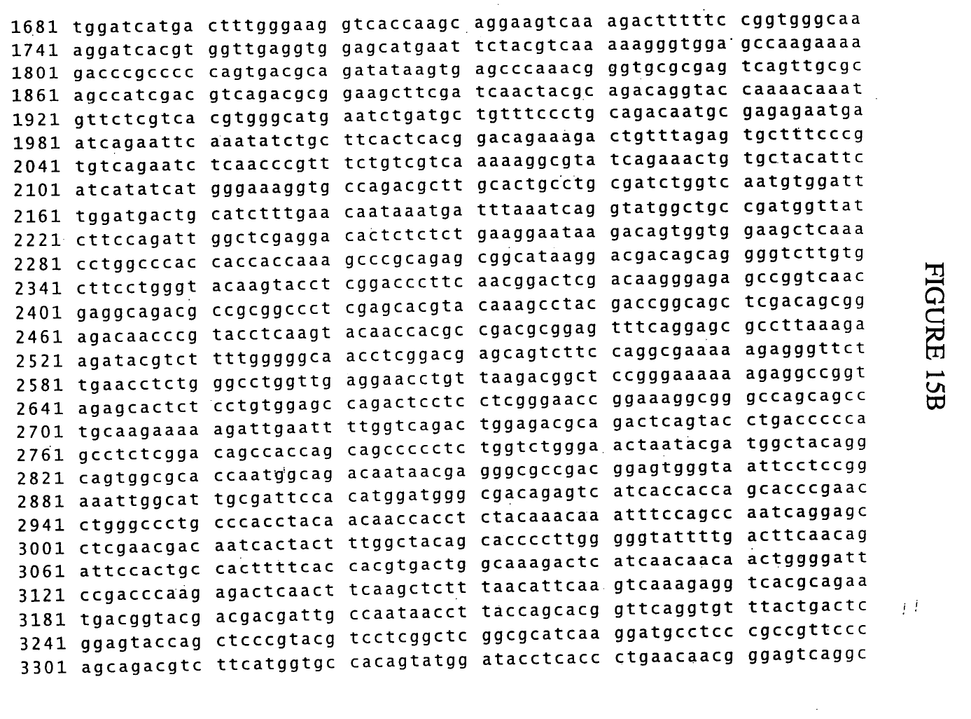

The first type of AAV Rep78 mutant, typified by Rep192^{HG} and phenotypically related Rep proteins that bind similarly to Rep192^{HG}, possesses enhanced DNA binding to at least one of a PV DNA, an AAV DNA or HIV DNA or an oncogene as compared to the wild-type AAV Rep78 protein's binding to these DNAs. This latter mutant type inhibits PV or other target genes by inhibiting the transcriptional activity or promoter of this virus or other target genes, and also inhibits the transcriptional activity or promoters that drive the expression of oncoproteins. Enhanced promoter binding directly correlates with enhanced promoter inhibition (See FIGS. 10, 14). Therefore, these latter mutants are useful in anti-PV and anti-cancer protocols, not only for treating PV, but also for inhibiting the oncogenic transformation of the cell in general. This mutant type is demonstrated by the Rep-192^{HG} and is disclosed in the present invention. But other similar mutants may be selected using the inhibition assays described in the present invention.

The second type of AAV Rep78 mutant is a mutant possessing weak or no DNA binding affinity to at least one DNA sequence obtained from a PV or an AAV, such as the promoter, AAV p5 or HPV-16 p97, when this binding affinity is compared to the binding of the wild-type AAV Rep78 protein. Although these mutants bind less strongly, if at all, to the selected DNA(s), these mutants have other functions that are intact, and therefore have enhanced ability to complement AAV functions, that results in the generation of higher levels of AAV DNA replication and/or AAV virion numbers. These increased levels of AAV DNA replication and virions are useful for generating more rAAV for gene therapy as compared to the wild-type Rep78. But these weak or no binding AAV Rep78 mutants, also are useful in treating PV by virtue of the negative impact that the presence of AAV has on the presence of PV. Thus, any mechanisms that would increase AAV numbers is expected to decrease PV. The Rep-77^{LG}, Rep-79^{FA} and the Rep-64^{LH}65^{TM} mutants are examples of this type of mutant and are disclosed in the present invention.

In another embodiment, the present invention is directed to AAV Rep78 mutants that include 1) truncated wild-type AAV Rep78 proteins, 2) wild-type AAV Rep78 proteins containing amino acid substitutions, 3) wild-type AAV Rep78 proteins containing internal amino acid deletions, and 4) wild-type AAV Rep78 proteins that contain combinations of these modifications. The AAV Rep78 mutant in one embodiment is a modified AAV Rep78 protein containing at least the minimum number of amino acids of the wild-type AAV Rep78 protein necessary to bind to at least one of the PV DNA, an oncogene or the AAV DNA, or HIV DNA, preferably the promoter region of these DNAs, to obtain enhanced inhibition of PV or oncoproteins or HIV. In the case of the oncogene encoding the oncoprotein, the proto-oncogene promoter is inhibited. In certain mutants, this modification could reduce the binding affinity. If enhanced binding is the property of the AAV Rep78 mutant, then the goal of the present invention is to select the smallest truncated AAV Rep78 mutants that are still capable of binding to the PV DNA, oncogene, AAV DNA, HIV DNA or a combination thereof, to inhibit the expression of PV, PV oncoproteins or other cancer related oncoproteins or HIV to inhibit transcription with the end result being the inhibition of the PV-associated disease or cancer or HIV-associated disease. Again, if enhanced binding to the promoter region is the goal, then the truncated AAV Rep78 mutant must still maintain the ability to bind to the promoter region of at least one of PV DNA or AAV DNA or oncogene or HIV DNA, and particularly bind to nucleotides 14-56 of the p97 of HPV-16. Truncated Rep 78 mutants, within the context of the present invention, include any deletion of amino acids of the wild-type Rep78 protein. These deletions may be serial deletions of amino acids from the N-terminus, middle or C-terminus of the protein or one or more amino acid deletions from different portions of the protein. A further embodiment is directed to a truncated AAV Rep78 mutant that is composed of at least two of these truncated wild-type AAV Rep78 linked to each other to form a multimer AAV Rep78 mutant. Protein fusions are performed by techniques known in the field or are made by recombinant techniques by linking the DNAs encoding the proteins with the resulting expression of a fusion protein.

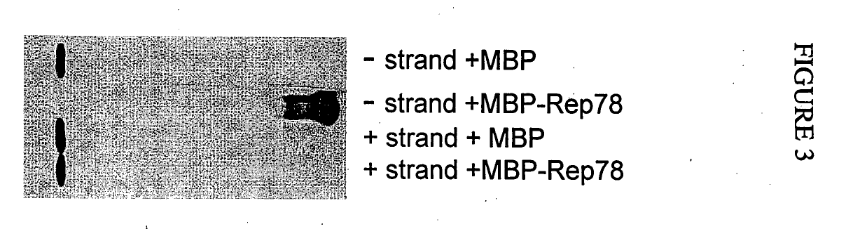

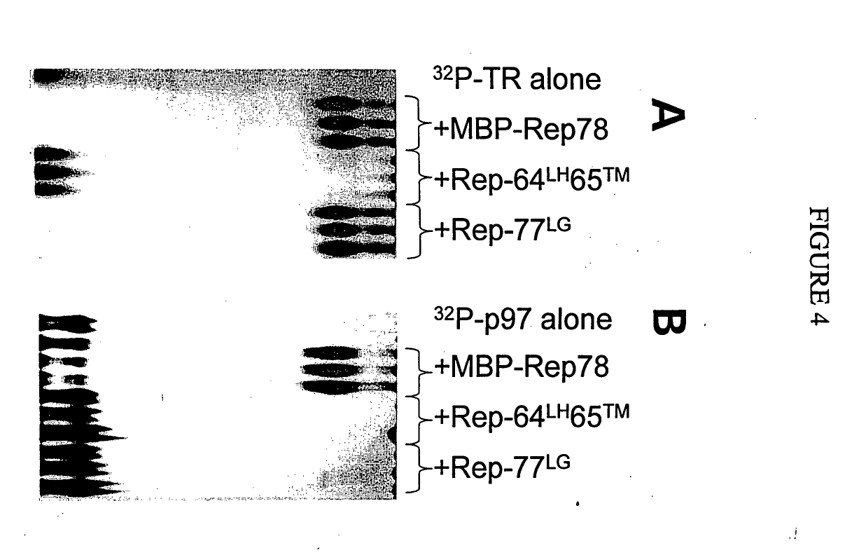

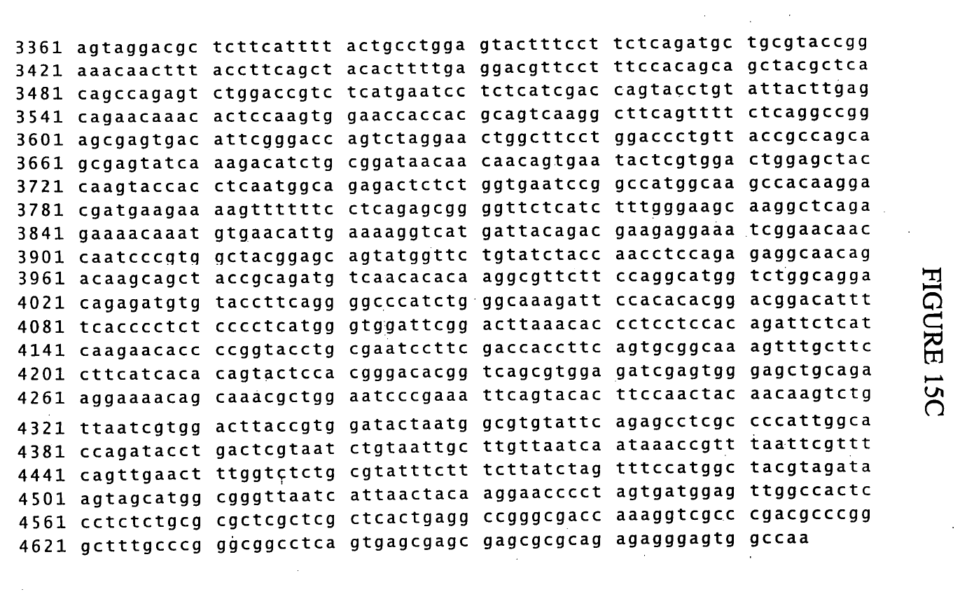

The present invention provides sufficient guidance to a person skilled in the art to obtain additional AAV Rep78 mutants that have modified binding affinities as compared to the wild-type AAV Rep78. The nucleic acid sequence is disclosed in Srivastava et al. J. Virol. 45:555 (1983) and is disclosed in FIG. 15A-C which is used to prepare mutants according to the present invention. AAV has been studied and the organization of the AAV genome is disclosed in Muzyczka, N. et al., Current Topics in Microbiology and Immunology 158: 97-129 (1992), particularly FIG. 3. The present invention discloses a sequence to which the AAV Rep78 binds to in FIG. 2 and discloses methods to test for the strength of the binding or the lack of binding to DNA sequences, preferably promoter regions. The present invention is intended to encompass other AAV Rep78 mutants that have modified DNA binding; for example, promoter binding affinities. Such methods would not require undue experimentation and are disclosed in the present invention or known to skilled persons.

In a further embodiment, the present invention is directed to a fusion protein comprising an AAV Rep78 mutant described above. The fusion protein envisioned in the present invention is one in which the tat protein of HIV is linked to the AAV Rep78 mutant or the wild-type AAV Rep78. Particularly, the tat protein of HIV type 1 is preferred for linking to the AAV Rep78 wild-type protein or mutants to provide a fusion protein for direct treatment of the Rep78 mutant and the tat protein of HIV to cells and tissues. The method circumvents the need for introducing the gene encoding the AAV Rep78 mutant via gene therapy. The tat-wild-type Rep78 or tat-Rep78 mutan, optionally as a fusion protein with MBP (E. coli maltose binding protein), is able to be actively taken up by cells. This method provides for these proteins containing tat to be taken up and allows AAV Rep78 and its mutants to be applied by cream or other forms of an external pharmaceutical compositions for dermatological application. Further, these fusion proteins also are applied by direct injection into the tumor sites for a potentially higher effectiveness or administered directly to the tumor if visible from the surface of the skin. Nagahara et al., Nature Medicine, 4(12): 1449 1998 discloses the use of tat in fusion proteins.

The present invention is also directed to a fusion protein comprising a maltose-binding protein (MBP) linked to said AAV Rep78 mutant. See Batchu et al., Biochem. Biophys. Res. Commun. 208: 714 (1995).

The present invention further is directed to pharmaceutical compositions comprising at least one AAV Rep78 mutant or AAV Rep78 protein as disclosed above in admixture with a pharmaceutically acceptable carrier. The preferred manner of treatment using these pharmaceutical compositions is subcutaneous injection or directly into the tumor or via topical administration using creams or lotions that may include lipofection where the mutant is carried in beads made of bipolar lipids. Pharmaceutical compositions and appropriate carriers for each of these modes of administration are known to persons skilled in the art and can be combined without undue experimentation with the AAV Rep78 proteins and mutants of the present invention. See, for example, Remington's Pharmaceutical Sciences: Drug Receptors And Receptor Theory, (18th ed.), Mack Publishing Co., Easton, Pa. (1990) These pharmaceutical compositions are administered to inhibit PV-associated diseases, cancer and HIV-associated diseases in patients having these diseases.

AAV as well as adenoviruses and retroviruses, are useful as gene transfer vehicles in gene therapy. Gene therapy techniques have been known for several years, and as discussed in Kay et al., Proc. Natl. Acad. Sci. USA, 94:12744 (1997), vehicles for gene transfer can be classified into two major classes: viral and nonviral vectors. Table 1 of Kay et al. discloses retroviruses, adenoviruses and adeno-associated viruses (AAV) as viral vehicles and liposomes as nonviral vehicles. The drawbacks of using retroviruses and adenoviruses are that they are large and as such, carry extra genes into the transduced cells, which encode additional proteins that likely will elicit an immune response from the patient. AAV is smaller in size and provides less complicated epitopes to the patient than retroviruses or adenoviruses. Both AAV and retroviruses have been demonstrated as being highly effective gene transmitters in immortalized cells. However, retroviruses are unable to transduce non-dividing cells. Furthermore, there is another very important difference between the two viruses in regard to their use as a human gene therapy vector. AAV is non-pathogenic. In contrast, retroviruses are pathogenic. This fact was recently emphasized in monkey bone marrow gene therapy experiments utilizing retrovirus based vectors, even minute amounts (10⁻⁶ of the vector) of contaminating wild-type retrovirus resulted in the most of the animals developing malignant lymphoma (Donahue et al., J. Experimental Medicine 176(4): 1125 (1992); Vanin et al., J. Virol. 68(7): 4241 (1994)). AAV actually has anti-cancer properties. Therefore, AAV is a suitable choice for transducing papilloma tumor cells, such as cervical cancer or other cancers with which HPV is associated (Hermonat et al, Proc. Natl. Acad. Sci. USA, 81: 6466 (1984)).

In a further embodiment, the present invention is directed to a DNA sequence encoding at least one AAV Rep78 mutant or AAV Rep78 protein described in this application. Additionally, the DNA sequence encoding an AAV Rep78 protein or a mutant thereof is further contained in a vector for delivery of the virus to the target cell in culture or in a patient's body that will benefit from treatment of AAV Rep78 or a mutant thereof. This vector is any known vector and for gene therapy is preferably an AAV vector but other known vectors, such as retroviruses, adenoviruses, pox viruses or liposomes are also useful in the present invention. This vector containing the DNA sequence is admixed with a pharmaceutically acceptable carrier for administration to a patient afflicted with a PV, PV-associated disease, cancer, HIV or HIV-associated disease. This method of treatment delivers DNA encoding AAV Rep78 protein or an AAV Rep78 mutant to the PV tumor of the patient so that AAV Rep78 DNA or its mutant DNA transduces the cells and is expressed by the affected cells causing an inhibition of PV production or oncoproteins. The vector is useful for gene transfer: ex vivo where cells are removed, genetically modified by transduction with the vector, and transplanted back into the same recipient or in vivo therapy accomplished by transfer of genetic materials directly into the patient's cells in the body. Methods of constructing and using such vectors are known in the art. Hermonat, et al., Proc. Natl. Acad. Sci. U.S.A. 81:6466 (1984); McLaughlin et al., J. Virology 62(6): 1963 (1988); Hermonat et al., FEBS Letters 407-:78 (1997); Liu et al., J. Inf. Cytok. Res. 20:21-30. (2000); Hermonat et al., J. Hum. Virol. 3: 113-124 (2000).

The present invention is further directed to a DNA construct comprising a DNA sequence encoding AAV Rep78 or a AAV Rep78 mutant operably linked to an inducible promoter, preferably a promoter that is induced by a molecule produced during PV infection or cancer, such as an oncoprotein. As discussed above, this DNA construct is useful for insertion into a DNA vector, such as an AAV vector, retrovirus, or other vectors as discussed above, for transfer into mammalian cells, when admixed with a pharmaceutically acceptable carrier for treating a patient and inhibiting PV-associated disease in the patient. Examples of such inducible promoters that are functional in the present invention are the BPV p89 promoter that is responsive to the PV E2 protein and the promoter of the E2 gene of adenovirus which is responsive to the HPV E7 oncoprotein. Other appropriate promoters inducible by specific PV proteins may be selected by persons skilled in the art.

The present invention also is directed to a DNA sequence comprising the full length AAV genome (FLAG) modified in that the AAV Rep78 gene is replaced with an AAV Rep78 mutant of the present invention.

The present invention further is directed to a DNA sequence comprising at least the minimum portion of the AAV genome sufficient to complement a defective rAAV, wherein the portion of the AAV genome is modified to replace the AAV Rep78 DNA sequence with the DNA sequence encoding the AAV Rep78 mutants described herein, such as the AAV Rep-77^{LG} and Rep-79^{FA} mutants, which possesses no DNA binding or weak DNA binding to the DNA sequence obtained from at least one of a PV or an AAV or an oncogene, preferably the promoter regions of these sources, that results in the generation of higher levels of rAAV DNA replication and virion numbers. This DNA sequence is useful in producing rAAV comprising transducing a susceptible mammalian cell with the minimum portion of the AAV genome sufficient to complement a defective rAAV as a complementor, and a DNA sequence encoding the rAAV to obtain increase numbers of rAAV or AAV. The rAAV is the AAV with all of the AAV genes removed except the TR sequences (terminal repeats), which renders the rAAV defective, and in need of a helper virus as a complementor, to provide the missing AAV genes for replication. The rAAV also contains a DNA sequence encoding a heterologous gene. The DNA sequence sufficient to complement a defective rAAV comprises at least the AAV lip-cap gene and the DNA sequence encoding an AAV Rep78 mutant that binds weakly or not at all to DNA sequences, preferably a promoter region from at least one PV, an oncogene, AAV or HIV.

The present invention further is directed to a method of inhibiting PV, PV-associated diseases, cancer, HIV or HIV-associated diseases comprising administering a pharmaceutical composition comprising an AAV Rep78 protein or a mutant thereof, to a patient afflicted with a PV-associated disease, wherein the AAV Rep78 protein or mutant binds to HPV DNA, particularly binds to nucleotides 14-56 of p97 of HPV-16, thereby inhibiting expression of HPV oncoproteins and/or binds to HIV DNA, for example, such as HIV promoter sequences TAR or nucleotides −54 to −34.

The present invention also discloses an AAV Rep78 regulation element comprising the nucleotides shown in the nucleotide sequence of FIG. 2, wherein this element provides the binding site for the AAV Rep78 protein, and particularly comprises about nucleotides 14-56 of the nucleotide sequence of FIG. 2. This present invention also discloses an AAV Rep78 regulatable promoter comprising the regulation element of the nucleotides shown in the nucleotide sequence of FIG. 2, wherein this element provides the binding site for the AAV Rep78 protein, and particularly comprises about nucleotides 14-56 of the nucleotide sequence of FIG. 2. This AAV Rep78 regulatable promoter comprises the regulation element described above and the remaining promoter sequences from a promoter other than the HPV-16 p97 promoter.

The following specific examples provide guidance to the skilled person regarding the methods useful in preparing and testing AAV Rep78 mutants for their capability and relative affinity in binding PV DNA and AAV DNA and testing the effects of that binding or lack thereof on transcription. These examples should by no means be interpreted as limiting the present invention to these disclosed examples but as a guide to select additional AAV Rep78 mutants that fall within the spirit of the present invention.

### Examples

The following experimental procedures are utilized in the present invention:

### Experimental Procedures

### AA V Rep78 Affinity Chromatography Selection of HPV-16 DNA Fragments

The Bam HI fragment containing the complete HPV-16 genome (without plasmid sequences, from pAT/HPV-16) was isolated by gel electrophoresis using the GeneClean II kit. This DNA was further digested with Pst I, Klenow labeled with α³²P-dCTP, and cleaned by phenol extraction and ethanol precipitation. The AAV Rep78 affinity chromatography was carried using 50 μgs of MBP-AAV Rep78 bound to 100 μls of amylose resin (New England Biolabs). Klenow labeled ³²P—HPV-16 DNA (2 μgs), digested by Pst I and Bam HI, were applied to the column in 100 μls of column buffer (20 mM Tris [pH 7.4], 200 mM NaCl, 1 mM EDTA, 1 mM DTT) and incubated for 15 minutes at room temperature. After washing the column twice with 1 ml of column buffer, the bound DNA was eluted with 100 μls of 1% SDS, 20 mM Tris pH7.5. The eluted products were then analyzed by PAGE (4%), dried and autoradiographed.

### DNA Substrates and the Electrophoretic Mobility Shift Assay (EMSA)

HPV-16 and MSV-LTR DNA substrates were generated by polymerase chain reaction (PCR) amplification. The AAV terminal repeat (TR) substrate was generated by the ligation of three separate synthetic oligonucleotides as described previously (Bishop, et al., FEBS Letters 397:97-100 (1996)). The TR was 5′ end labeled with polynucleotide kinase using ³²P ATP (5,000 Ci/mmol, Amersham). Single stranded DNA substrates were generated by asymmetric PCR amplification as previously described (Gyllensten, et al., Proc. Natl. Acad. Sci. USA 85:7652-7656 (1988)). EMSA was carried out as follows; approximately 1 ng of ³²P-labeled DNA substrate was incubated with increasing amounts of MBP-AAV Rep78 for 10 min. at room temperature in binding buffer [(25 mM HEPES KOH pH 7.5, 10 mM MgCl₂, 1 mM dithiothreiotol, 2% glycerol, 25 ug bovine serum albumin, 50 mM NaCl, 0.01% NP40 and 0.5 ug poly (dI-dC)]. Samples were electrophoresed in a 4% polyacrylamide gel (40:1 acrylamide and bis-acrylamide weight ratio) with 5% glycerol in 0.5×TBE buffer at 100V for about 3 hr. Gels were dried and autoradiographed at −70° C.

### Construction of AAV Rep78 Mutant Plasmids and Production of MBP-AAV Rep78 Chimeric Proteins

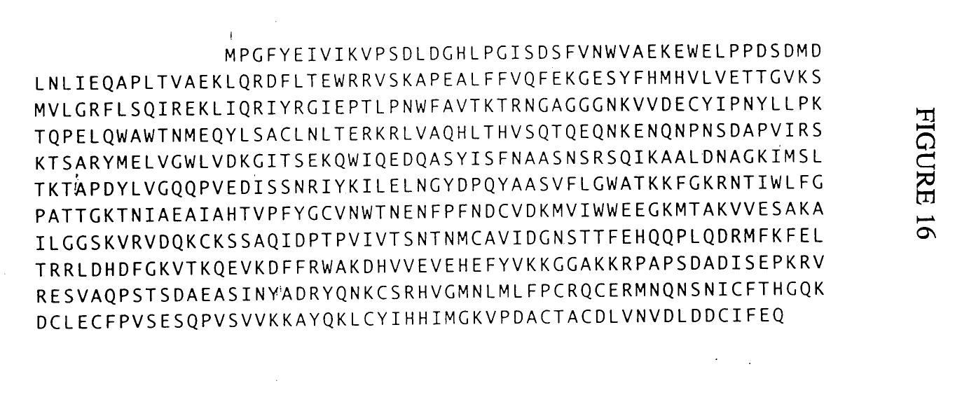

The construction of any of the AAV Rep78 mutants utilizes the know AAV Rep78 nucleic acid sequence as disclosed, for example, in FIGS. 15A-C and in Srivastava et al., J. Virol. 45:555 (1983). Further, the 621 amino acid wild-type Rep78 encoded by nucleotides 321-2183 (stop codon nucleotides 2184-2186) is disclosed in FIG. 16 and was known. The construction of the plasmid pMAL-Rep-64^{LH}65^{TM} from which mutant MBP-64^{LH}65^{TM} protein is produced, has been described previously (Batchu, et al., Biochem. Biophy. Res. Comm. 208:714-720 (1995), Batchu, et al., Biochem. Biophy. Res. Comm. 210:717-725 (1995)). The plasmid pMal-Rep-77^{LG} was similarly constructed using a different mutagenic oligonucleotide (5′-CCCCGGAGGCCGAATTCTTTGTGCAA) and the M13 based plasmid pALTER-AAV3 (containing all of the AAV genes). A second oligonucleotide created a Sph I restriction site immediate upstream of AAV Rep78 ORF. The mutations were initially characterized by the generation of a new restriction site and were further verified by DNA sequencing with Sequenase (U.S. Biochemicals) according to the manufacturer's recommendations. The mutant AAV Rep78 ORFs were then transferred into pMALc2 on an Sph I and Xho I fragment (nt. 321 to nt. 2233) to generate pMAL-Rep-64^{LH}65^{TM} and pMAL-Rep-77^{LG}. Both the mutant and wild-type fusion proteins with MBP were purified by affinity chromatography using amylose resin following kit directions (Protein Purification and Expression System, New England Biolabs). Fractions were collected and analyzed by SDS-polyacrylamide gels. Purified fractions were concentrated using Centricon 10 kDa cut-off membrane filters (Amicon). Routinely these procedures resulted in MBP-AAV Rep78 and 64^{LH}65^{TM} proteins of 70-90% purity with a yield of 20 μg/100 ml bacterial culture.

### Construction of Full Length AAV Genomes (FLAG) Carrying the AAV Rep78 Mutations

The Bsa I fragment (4.2 Kb, containing all of the AAV genes) from pALTER-AAV-64^{LH}65^{TM} and pALTER-AAV-77^{LG} were isolated and ligated to the Bsa I partial digestion fragment (4.9 Kb, containing pBR322 plus the AAV TRs) from pSM620 (Samulski, et al., Proc. Natl. Acad. Sci. USA 79:2077-2081 (1982)) to generate pFLAG-64^{LH}65^{TM} and pFLAG-77^{LG}. To insure that the mutations were transferred into the full length AAV background, the region of the mutation was once again sequenced as described above.

### In Vitro Transcription Analysis of p97 Promoter Activity

An HPV-16 p97-CAT DNA fragment was used as a template for transcription. The p97-CAT DNA fragment was generated by PCR amplification using primer 1 (5′ACAAGCAGGATTGAAGGCCA, HPV-16 nt 7043-7065) complementary to the p97 sequences) and primer 2 (5′ CATATCACCAGC TCACCGTC, nt 615-633 of pSV2CAT) complementary to the CAT sequences). The plasmid p16P (p97-CAT) was used as the original PCR template (Romanczuk, et al., J. Virol. 64:2849-2859 (1990)). This produced a 1.2 Kb product. A 25 μl reaction mixture contained 0.5 μg of DNA template, 20 mM HEPES pH 7.9, 5 mM MgCl₂ 100 mM KCl. 0.5 mM DTT, 20% glycerol, 25 μM [³²P] GTP, 400 μM ATP, CTP and UTP, and 8 units Hela nuclear extract (Promega, HPV positive cervical cancer) or 5 μg of T-47D nuclear extract (Geneka Corp., HPV negative breast ductal carcinoma). Reactions were incubated at 30° C. for 60 min, and then terminated by adding 175 μL of Stop Solution containing 300 mM Tris-HCl, pH 7.9, 0.5% SDS, 300 mM sodium acetate, 2 mM EDTA and 3 μg/ml tRNA. RNA was extracted with phenol-chloroform, precipitated with ethanol, and finally dissolved in 10 μL of formamide containing 0.1% each of xylene cyanol and bromophenol blue. Samples were analyzed on an 6% polyacrylamide, 7 M urea gel, died, analyzed by autoradiography. An p97-specific RNA product of approximately 300 bases results.

### Transient Chloramphenicol Acetyltransferase Assay

Transient CAT assays were carried out by calcium phosphate transfection of the p16P (p97-CAT) plasmid plus several AAV plasmids (amounts indicated within the Figure legenda). Forty eight hours after transfection cell extracts were prepared, equalized for protein content by spectroscopic analysis at 280 nm, and assayed as described previously (Romanczuk, et al., J. Virol. 64:2849-2859 (1990), Hermonat, P. L., Cancer Research 54:2278-2281 (1994)).

### Focus Formation Assay

Contact inhibited murine C127 mouse fibroblasts were calcium phosphate transfected with 3 μgs of the HPV-16/ras chimeric plasmid, pL67R (Schmitt, et al., Virology 172:73-81 (1989)) plus 6 μgs of either pSM620 (+AAV Rep78, wild-type), d110-37 (-AAV Rep78, large deletion)(Hermonat, et al., J. Virol. 51:329-333 (1984)), pFLAG-64^{LH}65™, or pFLAG-77^{LG}. The plasmid pL67R contains the EJ-H-ras coding sequences ligated in place of the E1 gene (Hermonat, P. L., Cancer Research 54:2278-2281 (1994)). Thus, pL67R contains three oncogenes (E6, E7, and ras), all of which are expressed from the p97 promoter. The cells were fed for two and one half weeks, fixed with formaldehyde and stained with methylene blue.

The following experiments were performed using the procedures described above:

### AA V Rep 78 Recognizes the p97 Promoter DNA

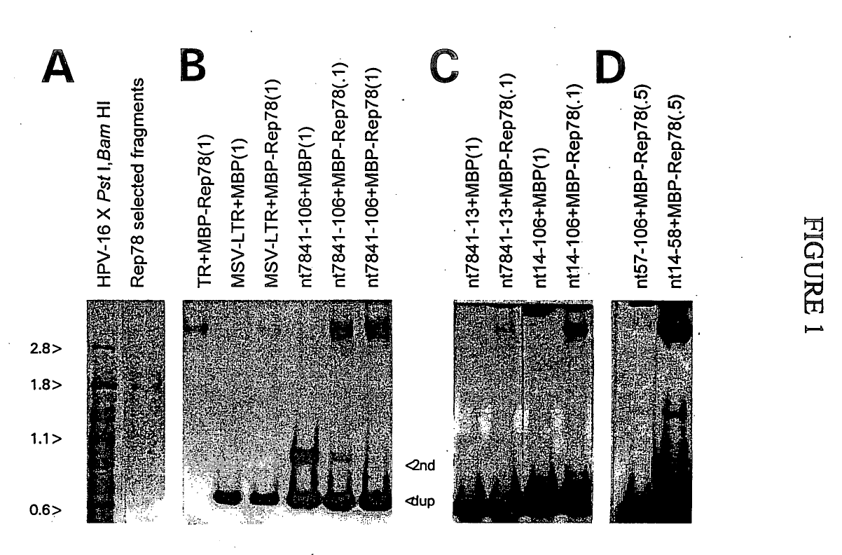

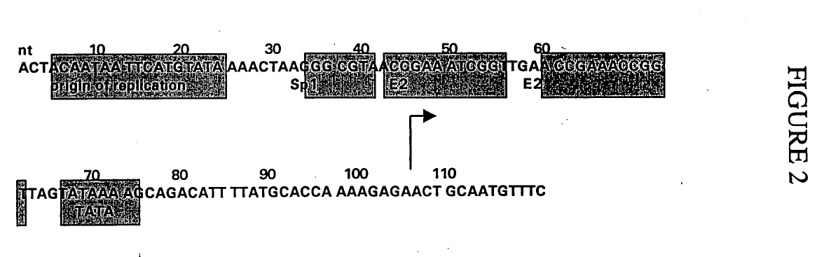

To observe if AAV Rep78 bound to HPV-16 DNA, AAV Rep78 affinity chromatography was utilized. MBP-AAV Rep78, bound to amylose resin, was incubated with ³²P-labeled HPV-16 DNA fragments generated from Pst I and Bam HI double digestion. After washing and elution the products were agarose gel electrophoresed adjacent to unselected HPV-16 fragments. As shown in FIG. 1A, although a few partial digestion products are present, it was clear that it was the 1.8 kb PstI fragment (nt 7003-875) of HPV-16 which was preferentially recognized by AAV Rep78.

Within the 1.8 kb fragment lies the long control region (LCR) of HPV-16 which contains central cis elements (origin of replication [ori], enhancers and promoters) essential for HPV-16 biological function. Furthermore, AAV Rep78 is a viral transcription factor known to bind promoter DNA (McCarty, et al., J. Virol. 74:4988-4997 (1994), Batchu, et al., Cancer Letters 86:23-31 (1994), Wonderling, et al., J. Virol. 71:2528-2534 (1996)). Thus, it is reasoned that AAV Rep78 might be targeting the ori/p97 region within this fragment due to AAV Rep78's known modulation of the HPV-16 p97 and HPV-18 p105 promoters (Hermonat, P. L., Virology 172:253-261 (1989), Hermonat, P. L., Cancer Research 54:2278-2281 (1994), Hermonat, et al., Gynecologic Oncology 66:487-494 (1997), Horer, et al., J. Virol. 69:5485-5496 (1995)), and of BPV-1 DNA replication (Hermonat, P. L. Virology 189:329-333 (1992)). To map the region of binding sequentially smaller substrates from this region were tested for recognition by AAV Rep78 (FIGS. 1B-C) by EMSA analysis. In FIG. 1B AAV Rep78 was shown to strongly recognize the HPV-16 sequences from nt 7814-106 (p97), while it does not significantly recognize a similarly sized analogous fragment from the MSV-LTR. These data clearly indicate that there is a specific recognition of the p97 DNA well above non-specific binding. As mentioned earlier, AAV Rep78 is able to inhibit expression from p97, but has little effect on the MSV-LTR (Hermonat, P. L., Cancer Research 51:3373-3377 (1991)). Thus, AAV Rep78 binding of promoter DNA may be associated with an ability to regulate p97 expression. In FIG. 1C, the target sequence was further defined to be in the 3′ half of this region (nt 14-106, hereafter referred to as “p97”). Finally, in FIG. 1D, a strong target sequence for AAV Rep78 binding is shown to be contained within nt 14-56. FIG. 2 shows the sequences of this region. Note that the nt 14-56 sequences contain an intact E2 binding motif and an Sp1 binding motif. These sequences also partially overlap the HPV-16 ori and E1 binding regions.

The E2 motifs are interrupted palindromes and can potentially form hairpin structures. There are also other interrupted palindromic sequences present within the p97 promoter region (nt 14-106). The DNA substrate which has formed some degree of secondary structure (2ndS) was observed in the EMSA gels as an elevated band over the lower simple duplex (dup) DNA substrate. It is this higher band which is the preferred substrate for AAV Rep78 recognition as demonstrated most clearly in FIG. 1B. This preference for DNA substrates with secondary structure has also been seen in AAV Rep78 recognition of the TAR region DNA of HIV type 1 (Batchu, et al., FEBS Letters 367:267-271 (1995)). In order to test the possibility that AAV Rep78 was recognizing secondary structure, the + and − strands of the p97 sequence were separately generated by single sided PCR. Such single stranded DNA should naturally form secondary structure as single stranded RNA does. The ³²P-labeled + and − strands were compared by EMSA for AAV Rep78 recognition as shown in FIG. 3. As can be seen the − strand was strongly recognized by AAV Rep78, while the + strand was not. These data suggest that AAV Rep78 may be recognizing both secondary structure and the specific sequence of the DNA. Two AAV Rep 78 amino acid substitution mutants are defective for binding p97 promoter DNA AAV Rep78 mutant proteins with specific amino acid substitutions for study in dissecting AAV Rep78's functions and domains are generated. The mutant AAV Rep78 proteins were produced as fusions with the maltose binding protein (MBP) as previously described (Batchu, et al., Biochem. Biophy. Res. Comm. 208:714-720 (1995), Batchu, et al., Biochem. Biophy. Res. Comm. 210:717-725 (1995)). During the characterization of one MBP-AAV Rep78 mutant protein, Rep-77^{LG} (substituting a glutamine for a leucine at amino acid 77), it was found to be able to bind an AAV terminal repeat (TR) DNA substrate at levels comparable to wild-type MBP-AAV Rep78, as shown in FIG. 4A. In contrast Rep-77^{LG} was defective in recognizing the p97 promoter as shown in FIG. 4B. In these experiments the wild-type MBP-AAV Rep78 and Rep-64^{LH}65^{TM} proteins served as the positive and negative controls, with Rep-64^{LH}65^{TM} being unable to bind any DNA substrate thus far assayed by EMSA (Batchu, et al., Biochem. Biophy. Res. Comm. 210:717-725 (1995), Kokorina, et al., J. Hum. Virol 1:441-450 (1998)). Thus, both Rep-64^{LH}65^{TM} and Rep-77^{LG} were defective for binding p97. However, Rep-77^{LG} was particularly interesting as it was able to distinguish between the TR and p97 substrates. Thus, the mechanism of recognition used by AAV Rep78 to bind the AAV TR was different, at least in part, than that for recognizing p97.

### AAV Rep78 Mutants Defective for Binding p97 are Also Defective for Inhibition of p97 Promoter Activity

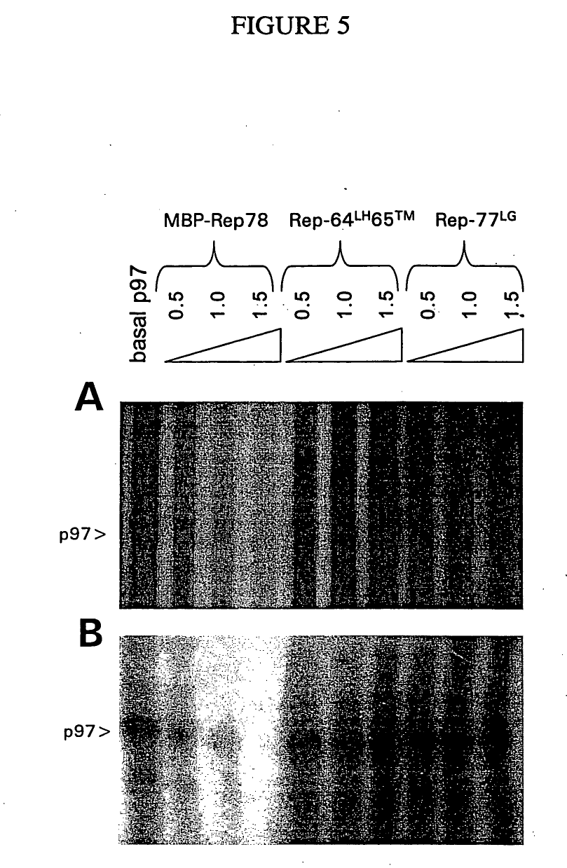

The effects of AAV Rep78 protein DNA binding on p97 promoter activity was observed. In vitro transcription was used to study these protein affects. Thus, various amounts of all three proteins (wt, Rep-64^{LH}65^{TM}, and Rep-77^{LG}) were added to HeLa and T-47D cell nuclear extracts and containing a p97 DNA template. The experiments were done four times using HeLa extracts and twice using T-47D extracts, all giving similar results. Representative experiments are shown in FIG. 5. As seen, the addition of increasing amounts of maltose-binding protein (MBP)-AAV Rep78 inhibited RNA initiation from the p97 promoter in a dosage dependent manner in both the HeLa and T-47D extracts. In sharp contrast to MBP-AAV Rep78, the addition of increasing amounts of the AAV Rep78 mutants, AAV Rep-64^{LH}65^{TM} and AAV Rep-77^{LG}, protein had little or no effect on p97 promoter activity in both cell types. Thus, both AAV Rep78 mutants defective for binding p97 DNA were also defective for inhibition of p97 promoter activity.

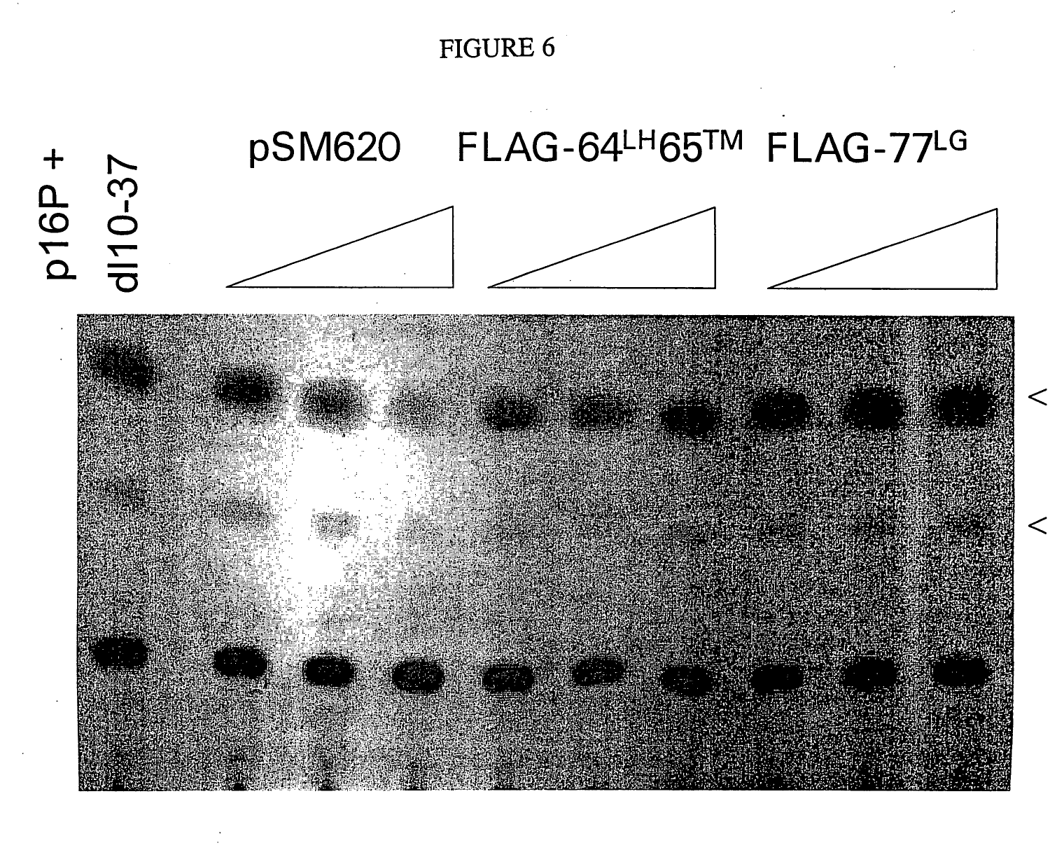

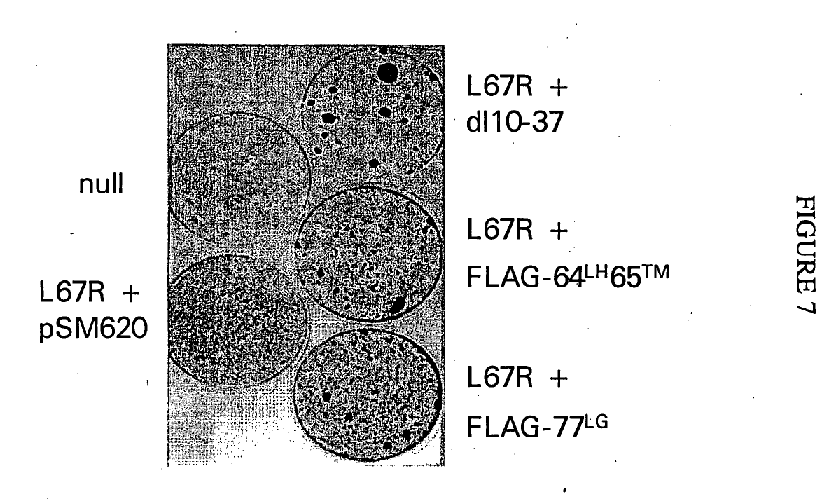

A CAT assay was also utilized to further verify AAV Rep78's effects upon the p97 promoter and the results are shown in FIG. 6. SW13 breast cancer cells were calcium phosphate transfected with the 4 μgs of plasmid p16P which contains the CAT coding sequences expressed from the HPV-16 p97 (Romanczuk, et al., J. Virol. 64:2849-2859 (1990)) plus different amounts of one of four AAV plasmids. FLAG plasmids, pSM620 (wild-type AAV Rep78), FLAG-64^{LH}65^{TM}, and FLAG-77^{LG} were co-transfected with p16P. As indicated by their names the FLAG plasmids contained the same mutated AAV Rep78 sequences as were present in the pMal-based plasmids used to generate the mutant proteins. The basal expression control plate was transfected with the AAV plasmid d110-37 which contains a large deletion within the AAV Rep78 sequences and served as a negative control. Cotransfection with increasing doses of pSM620 (2, 4, and 8 μgs, wild-type AAV Rep78) served as a positive control for inhibition. Mutant FLAG-64^{LH}65^{TM} and FLAG-77^{LG} were similarly cotransfected. Two days after transfection cellular extracts, equalized for protein content, were assayed for CAT activity. Note that pSM620 was able to inhibit p97 activity, while FLAG-64^{LH}65^{TM} and FLAG-77^{LG} were defective. The results shown in FIG. 6 demonstrate, similar to the in vitro transcription assays, that AAV Rep78 (pSM620) inhibits p97 activity while FLAG-64^{LH}65^{TM} and FLAG-77^{LG} were defective and did not inhibit p97 activity.

### AA V Rep 78 Mutants Defective for Binding p97 are Also Defective for Full Inhibition of HPV-16-Induced Oncogenic Transformation

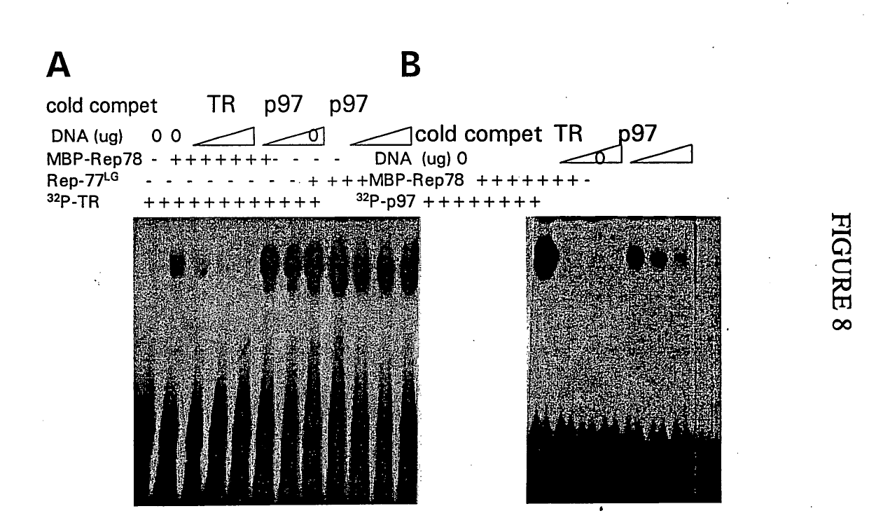

To further observe the biological significance of AAV Rep78-p97 interaction, the AAV Rep78 mutants (Rep-77^{LG} and Rep-64^{LH}65^{TM}), which were unable to bind p97 and unable to inhibit p97 transcription, were analyzed for their ability to inhibit HPV-16 p97-directed oncogenic transformation. The FLAG-77^{LG} and FLAG-64^{LH}65^{TM} mutant genomes were then tested in a C127 cell-based, HPV-16-induced focus formation assay. An HPV-16/ras chimeric plasmid, pL67R, in which the E1 gene is replaced by the EJ-H-ras coding sequences was utilized (Hermonat, P. L., Cancer Research 54:2278-2281 (1994), Hermonat, et al., Gynecologic Oncology 66:487-494 (1997)). This construct, expressing three oncoproteins from the p97 promoter, has higher transforming activity than HPV-16 alone (Hermonat, P. L., Cancer Research 54:2278-2281 (1994)). d110-37 (AAV Rep78 negative) and pSM620 served as the negative and positive controls, respectively. Plates of C127 cells were calcium phosphate transfected with pL67R (3 μgs) plus one of the indicated AAV plasmids (6 μgs). After 2.5 weeks the cells were formaldehyde fixed, methylene blue stained, and the foci counted. The results are shown in FIG. 7. Note that both FLAG-64^{LH}65^{TM} and FLAG-77^{LG} were defective when compared to pSM620 (wild-type AAV Rep78) for inhibiting pL67R. Two additional transfection sets gave similar results. As transformation by L67R is dependent on the p97 promoter, these data strongly suggest that the AAV Rep78-p97 interaction is important for AAV Rep78's inhibitory ability.

### AAV Rep78-p97 Interaction is not as Strong as AAV Rep78-TR Interaction

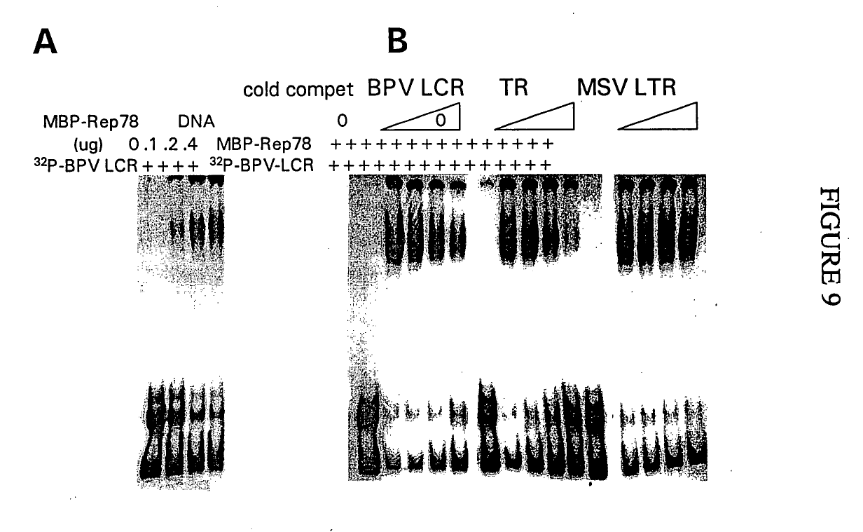

To compare the affinities of AAV Rep78 for the AAV TR and HPV-16 p97 DNA substrates a series of competitive EMSA experiments were undertaken. As shown in FIG. 8A unlabeled TR DNA competitor was able to strongly inhibit wild-type AAV Rep78-³²P-TR interaction, while unlabeled p97 DNA (nt 14-106) competitor was not. Unlabeled p97 DNA competitor was also not able to inhibit Rep-77^{LG}-TR interaction. In FIG. 8B it is shown that both unlabeled TR and p97 competitors are able to successfully inhibit wild-type AAV Rep78-³²P-p97 interaction, but that the TR is the more effective competitor. These data are consistent with AAV Rep78 having a higher affinity for the AAV TR (its natural substrate with GCTC³) than the HPV-16 p97 promoter (lacking GCTC motifs). These data are also consistent with the interpretation of the other data, FIG. 5, that the mechanism of AAV Rep78 recognition of p97 DNA is different than AAV Rep78 recognition of TR DNA.

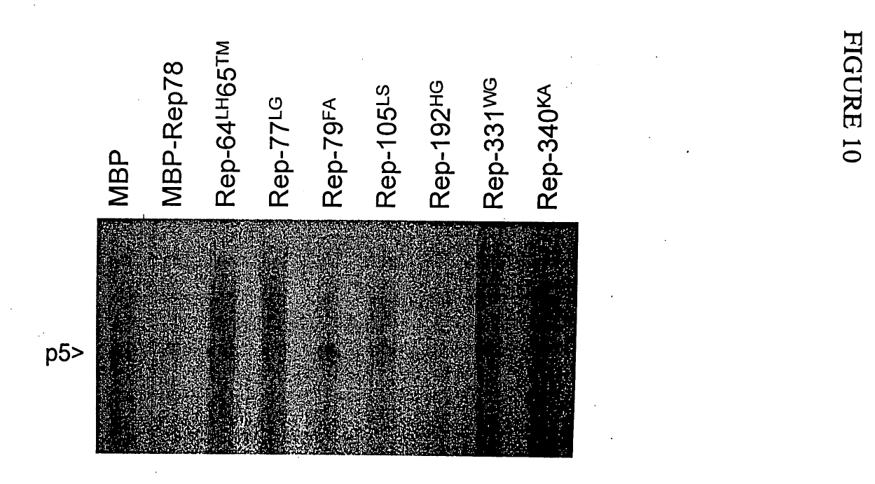

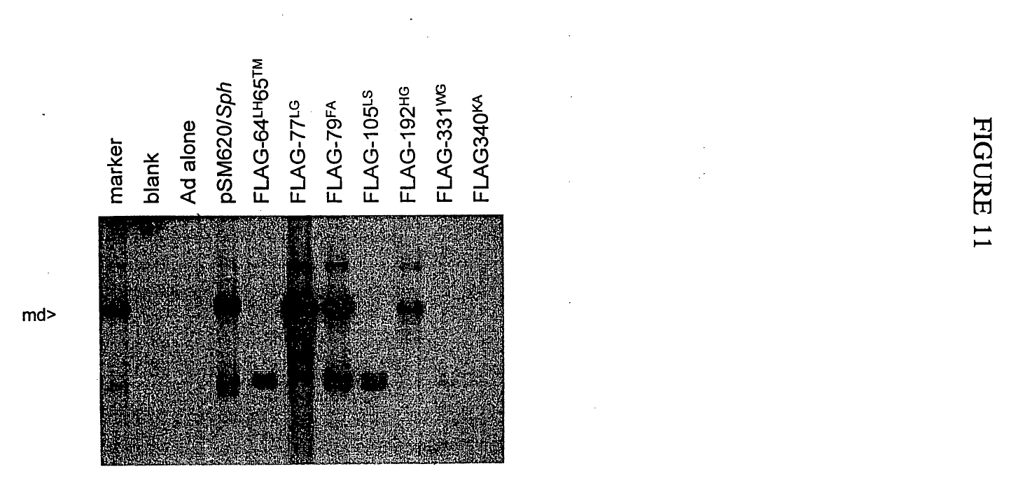

The BPV-1 LCR also contains multiple E2 motifs within E6 promoter (p89). Thus, an assay was performed to determine whether AAV Rep78 might bind to a sequence (nt 7758-7930) from p89 which contains two such motifs in close proximity. The EMSA results, shown in FIG. 9A demonstrate that AAV Rep78 is able to recognize and bind E2 motif DNA from BPV-1 p89. FIG. 9B shows that, as with the AAV Rep78-p97 interaction, both the unlabeled BPV-1 p89 and the AAV TR DNAs were able to compete against AAV Rep78-³²P-BPV-1 LCR interaction, however TR DNA was the better competitor.

### AAV Rep78 Mutants Effect on the AAV p5 Promoter

FIG. 10 provides an example of AAV Rep78 mutants, Rep-77^{LG} and Rep-79^{FA} that are unable to bind to the AAV p5 promoter and unable to inhibit the p5 promoter by in vitro transcription. The promoter, p5, is an important promoter as it expresses AAV Rep78 itself, which is critical for AAV replication. This mutant also is not able to bind to HPV-16 p97. The present invention shows that this inability to bind results in higher levels of AAV genome replication and virion production. The Rep-192^{HG} is able to inhibit p5 (AAV promoter) better than wild-type AAV Rep78, Rep-77^{LG} and Rep79^{FA}, which do not inhibit p5 at all. All data indicates that the mutant inhibition of AAV p5, HPV-16 p97 and HIV-1 long terminal repeat are similar. Additionally, the level of p5 transcription mimics and supports the level of DNA replication of these mutants as FLAGs for wild-type 620, Rep-77^{LG} and Rep-192^{HG}.

The present inventor discovered that by including this particular mutation in a full length AAV genome (FLAG-77^{LG}), he was able to obtain replication levels of 150% above wild-type AAV-2, which makes it useful as a complementor for generating recombinant AAV. FIG. 11 shows that FLAG-77^{LG} replicates at higher levels and FLAG-192^{HG} replicates at slightly depressed levels compared to wild-type AAV Rep78 (pSM620/Sph). Although FLAG-192^{HG} does not show a very low replication level in the experiment shown in FIG. 11 but over 5 experiments, FLAG-192^{HG} clearly replicates at lower levels than wild type pSM620. Another AAV Rep78 mutant that is unable to bind to p97 is Rep-64^{LH}65^{TM}.

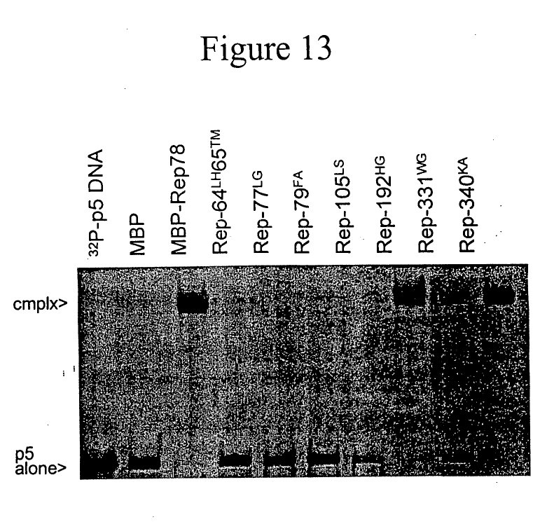

An example of an AAV Rep78 mutant that has enhanced binding as well as different binding to the AAV TR (terminal repeat) and the p5 is the Rep-192^{HG} as shown by a higher sized band on the EMSA in FIG. 12 and FIG. 13 as compared to the wild-type AAV Rep78 (MBP-Rep78), which makes it useful for anti-cancer protocols of treatment. FIG. 12 shows that Rep192^{HG} binds TR DNA in a different higher order complex and with higher affinity. The FLAG-192^{HG} replicates at lower levels than wild-type AAV Rep78, which is consistent with increased self-inhibition. AAV Rep192^{HG} is a super binder, and therefore, a super inhibitor, as shown by the higher and darker band in the Rep192^{HG} lane representing a protein-DNA complex.

The present invention discloses that the AAV Rep78 protein meaningfully binds to the p97 promoter of HPV-16. This binding, although not as strong as Rep78-AAV TR interaction, is clearly above the so-called “non-specific” DNA-binding ability of Rep78 as determined by the experiments presented in FIG. 1. The importance of this interaction is supported by the finding that two AAV mutants (FLAG-64^{LH}65^{TM} and FLAG-77^{LG}), whose Rep78 proteins are unable to recognize the HPV-16 LCR p97 target, are defective for in vitro transcriptional inhibition of p97 and for the inhibition of HPV-16 directed oncogenic transformation. It should also be noted that Rep78 is unable to significantly affect expression from the MSV-LTR (18) and Rep78 is also unable to bind partial (Batchu, et al., Cancer Letters 86:23-31 (1994), Kokorina, et al., J. Hum. Virol 1:441-450 (1998)) or the full length sequence of this promoter (FIG. 1B).

FIG. 14 shows the inhibition of HPV-16 by AAV Rep78 mutants. pL67R (HPV16+ras) and each of the indicated AAV plasmids were cotransfected (4 ug each) into contact inhibited C127 cells and allowed to grow for 2½ weeks, and oncogenic foci counted. Note that inhibition of HPV-16-induced oncogenic foci roughly matches the DNA binding ability of the Rep78 protein. The results show that Rep-192^{HG} inhibits HPV-16 oncogenic transformation and p97 promoter expression better than 620sph (wild-type) Rep 78, which demonstrates the anti-oncogenic properties of this Rep78 mutant.

One previous relevant study undertaken by Horer et al., J. Virol. 69:5485-5496 (1995) attempted to identify the cis-responsible element within the analogous p105 promoter of HPV-18. Their results, obtained by the deletion and mutation of sequences along the length of the p105 promoter, were inconclusive for finding a specific responsible element. Their interpretation of this data was that the mechanism of inhibition was complex and involved multiple components. The data disclosed herein may suggest such complexity, for although the phenotypes of mutants Rep-64^{LH}65^{TM} and Rep-77^{LG} are quite strong in FIG. 5, it is also clear that they are still able to mildly inhibit oncogenic transformation in FIG. 7 (only small foci were generated). It is reasonable to suggest that Rep78's protein-protein interactions are likely also involved in these other inhibitory pathways. In addition to Rep78's ability to meaningfully interact with transcription factors, in preliminary experiments, Rep78-E7 oncoprotein interaction by both Western blot, affinity chromography, and yeast GAL4 two-hybrid cDNA analyses has been observed (Zhan and Hermonat, unpublished).

The fact that Rep78 binds HPV-16 p97 is surprising as this target sequence contains no GAGC motifs, the core sequence of most Rep78 DNA recognition. Rep78 binding to promoter sequences has been observed before, including the AAV p5 promoter (McCarty, et al., J. Virol. 74:4988-4997 (1994)), the c-H-ras promoter (Batchu, et al., Cancer Letters 86:23-31 (1994)), the human immunodeficiency virus long terminal repeat TAR region (Batchu, et al., FEBS Letters 367:267-271 (1995), Kokorina, et al., J. Hum. Virol 1:441-450 (1998)), and the cytomegalovirus immediate early promoter (Wonderling, et al, J. Virol. 71:2528-2534 (1996)). All of these promoters have GAGC (or GCTC) motifs, and binding by the large Rep proteins (proteins 78/68) appears to be required for the inhibition of the AAV p5 promoter (Kyostio, et al., J. Virol. 69:6787-6796 (1995)) and the HIV-LTR (Kokorina, et al., J. Hum. Virol 1:441-450 (1998)). In experiments using Rep78 affinity selection of random DNA sequences the consensus target sequence contained a duplex GAGC motif (Chiorini, et al., J. Virol. 69:7334-7338 (1995)). However a small subset of these selected sequences had no GAGC motifs. Thus, there is a precedence for Rep78 binding DNA without GAGC motifs. However, none of these sequences have significant homology with p97. Furthermore, none of the random selected sequences contain interrupted palindromes as are present in p97. It has also been observed that Rep78 binds a region within the BPV-1 LCR, which contains two E2 motifs, and it is believed that these motifs are the specific target for Rep78 recognition. AAV's own TRs, Rep78's natural and favored substrate, also have significant secondary structure. This further strengthens the argument that such structures (E2 or otherwise) are involved in Rep78 recognition of p97. However, the phenotype of Rep-77^{LG} suggests that Rep78's recognition of AAV TR DNA is different than its recognition of p97. The finding that Rep78 specifically recognizes the negative strand of the p97 promoter is novel (FIG. 3). These data further indicate that Rep78 discriminates between single stranded DNA substrates, by sequence, as it does for double stranded substrates. Finally, Rep78 binding to p97 may also affect the E1-E2 complex from binding the origin of replication which is located just upstream (Sedman, et al. EMBO J. 15:5085-5092, 1996 (1996), Yasugi, et al., J. Virol. 71:891-899 (1997), Sarafi, et al., Virology 211:385-396 (1995)). Rep78 is known to inhibit BPV-1 DNA replication (Hermonat, P. L. Virology 189:329-333 (1992)). Rep 79^{FA} has a similar phenotype to Rep77^{LG} in that it does not bind to the p5 and p97 promoter sequences very well but unlike Rep77^{LG}, Rep79^{FA} does not bind to TR DNA.

All of the publications cited herein are incorporated in their entirety by reference as is an article entitled “Binding of the human papillomavirus type 16 p97 promoter by the adeno-associated virus Rep78 major regulatory protein correlates with inhibition” by Zhan et al., J. Biol. Chem. 274: 31619-31624 (1999).

We claim:

1. An AAV Rep78 mutant comprising an AAV Rep78 modified protein that possesses different biochemical and biological functions as compared to the wild-type AAV Rep78 protein.

2. The AAV Rep78 mutant of claim 1, wherein said AAV Rep78 modified protein binds to at least one DNA sequence obtained from one or more of a papillomavirus, an AAV, an oncogene or a HIV differently as compared to the binding of said wild-type AAV Rep78 protein.

3. The AAV Rep78 mutant of claim 2, wherein said different DNA binding is selected from the group consisting of no DNA binding, weak DNA binding and enhanced DNA binding as compared to the binding of said wild-type AAV Rep78 protein.

4. The AAV Rep78 mutant of claim 3, wherein said mutant having no DNA binding or weak DNA binding to said DNA sequence obtained from at least one of a papillomavirus, an AAV, an oncogene or a HIV that results in the generation of higher levels of AAV DNA replication and virion numbers.

5. The AAV Rep78 mutant of claim 3, wherein said mutant having enhanced DNA binding to said DNA sequence obtained from at least one of a papillomavirus or an oncogene that results in enhanced inhibition of at least one of a papillomavirus or an oncoprotein.

6. The AAV Rep78 mutant of claim 2, wherein said mutant is selected from the group consisting of a truncated wild-type AAV Rep78 protein, a wild-type AAV Rep78 protein containing amino acid substitutions, a wild-type AAV Rep78 protein containing internal amino acid deletions, and a combination thereof.

7. The AAV Rep78 mutant of claim 6, wherein said mutant is a truncated AAV Rep78 protein containing at least the minimum number of amino acids of the wild-type AAV Rep78 protein necessary to bind to said DNA sequence to obtain enhanced inhibition of a papillomavirus or an oncogene.

8. The AAV Rep78 mutant of claim 7, wherein said DNA sequence to which said mutant binds is a promoter region of said papillomavirus, said AAV or said oncogene.

9. The AAV Rep78 mutant of claim 8, wherein said papillomavirus promoter region is nucleotides 14-56 of p97 of HPV-16.

10. The AAV Rep78 mutant of claim 7, comprising at least two truncated wild-type AAV Rep78 linked to form a multimer AAV Rep78 mutant.

11. The AAV Rep78 mutant of claim 4, wherein said mutant is AAV Rep-77^{LG}, AAV Rep-79^{FA}.

12. The AAV Rep78 mutant of claim 5, wherein said mutant is AAV Rep-192^{HG}.

13. A fusion protein comprising the AAV Rep78 protein or said AAV Rep78 mutant of claim 1.

14. The fusion protein of claim 13, further comprising the tat protein of HIV linked to said AAV Rep78 mutant.

15. The fusion protein of claim 14, wherein said tat protein is the tat protein of HIV-1.

16. The fusion protein of claim 13, further comprising a maltose-binding protein (MBP) linked to said AAV Rep78 protein or said AAV Rep78 mutant.

17. The fusion protein of claim 16, further comprising the tat protein of HIV linked to said AAV Rep78 mutant.

18. The fusion protein of claim 17, wherein said tat protein is the tat protein of HIV-1.

19. A pharmaceutical composition comprising at least one AAV Rep78 mutant or said AAV Rep78 protein according to claim 1, in admixture with a pharmaceutically acceptable carrier.

20. A method of treating a papillomavirus-associated disease, cancer or a HIV-associated disease comprising administering said pharmaceutical composition of claim 19 to a patient afflicted with a papillomavirus-associated disease, cancer or a HIV-associated disease.

21. A DNA sequence encoding at least one AAV Rep78 mutant, AAV Rep78 protein or a fusion protein according to claim 1.

22. The DNA construct comprising said DNA sequence of claim 21 and a vector.

23. The DNA construct of claim 22, wherein said vector is selected from the group consisting of an AAV, a rAAV, a retrovirus, an adenovirus and a liposome.

24. The DNA construct of claim 22, further comprising an inducible promoter operably linked to said DNA sequence.

25. The DNA construct of claim 24, wherein said vector is selected from the group consisting of an AAV, a rAAV, a retrovirus, an adenovirus and a liposome.

26. The DNA construct of claim 24, wherein said inducible promoter is controlled by a viral protein or an oncoprotein.

27. A pharmaceutical composition comprising at least one of the DNA sequences of claim 21 in admixture with a pharmaceutically acceptable carrier.

28. A pharmaceutical composition comprising at least one of the DNA sequences of claim 22 in admixture with a pharmaceutically acceptable carrier.

29. A pharmaceutical composition comprising at least one of the DNA sequences of claim 24 in admixture with a pharmaceutically acceptable carrier.

30. A method of inhibiting a papillomavirus-associated diseases, cancer or a HIV-associated disease comprising administering said pharmaceutical composition of claim 28 to a patient afflicted with a papillomavirus-associated disease, cancer or a HIV-associated disease.

31. A method of inhibiting a papillomavirus-associated disease, cancer or a HIV-associated disease comprising administering said pharmaceutical composition of claim 29 to a patient afflicted with a papillomavirus-associated disease, cancer or a HIV-associated disease, wherein said inducible promoter is controlled by the expression of a papillomavirus protein, an oncoprotein or an HIV-associated protein in said patient as a result of said disease or cancer.

32. A DNA sequence comprising the full length AAV genome modified to replace the AAV Rep78 DNA sequence with said DNA sequence encoding said AAV Rep78 mutant of claim 21.

33. A DNA sequence comprising at least the minimum portion of the AAV genome sufficient to complement a defective rAAV, wherein said portion of the AAV genome is modified to replace the AAV Rep78 DNA sequence with said DNA sequence encoding said AAV Rep78 mutant of claim 21.

34. The DNA sequence of claim 33, wherein said DNA sequence encoding said AAV Rep78 mutant encodes an AAV Rep78 mutant that has no DNA binding or weak DNA binding to said DNA sequence obtained from at least one of a papillomavirus or an AAV or an oncogene that results in the generation of higher levels of AAV DNA replication and virion numbers.

35. A method of producing recombinant AAV (rAAV) comprising transducing a susceptible mammalian cell with a DNA sequence of claim 34 and a DNA sequence encoding said rAAV to obtain rAAV virions.

36. The method of claim 35, wherein said DNA sequence is sufficient to complement a defective rAAV comprises at least the AAV lip-cap gene and said DNA sequence encoding an AAV Rep78 mutant.

37. The method of claim 35, wherein said rAAV comprises a DNA sequence encoding a heterologous gene.

38. A method of treating a papillomavirus-associated disease, cancer or a HIV-associated disease comprising administering a pharmaceutical composition comprising a AAV Rep78 protein to a patient afflicted with a papillomavirus-associated disease, cancer or a HIV-associated disease, wherein said AAV Rep78 protein binds to a papillomavirus promoter that controls the expression of a papillomavirus oncoprotein or binds to a promoter that controls the expression of an oncoprotein.

39. The method of claim 38, wherein said AAV Rep78 binds to nucleotides 14-56 of p97 of HPV-16 inhibiting expression of HPV.

40. An AAV Rep78 regulation element comprising the nucleotides shown in the nucleotide sequence of FIG. 2, wherein said element provides the binding site for the AAV Rep78 protein.

41. The AAV Rep78 regulation element of claim 40 comprising about nucleotides 14-56 of the nucleotide sequence of FIG. 2.

42. An AAV Rep78 regulatable promoter comprising the regulation element of claim 40 and the remaining promoter sequences from a promoter other than the HPV-16 p97 promoter.

43. An AAV Rep78 regulatable promoter comprising the regulation element of claim 41 and the remaining promoter sequences from a promoter other than the HPV-16 p97 promoter.

44. A method of selecting an AAV Rep78 mutant that possesses different biochemical and biological functions as compared to the wild-type AAV Rep78 protein, wherein said method comprises:
contacting an AAV Rep78 mutant of claim 1 with at least one DNA sequence obtained from one or more of a papillomavirus, an AAV, a HIV or an oncogene for a period of time to allow binding of said mutant to said DNA; and
determining said binding of said mutant to select said mutant that binds differently to said DNA than said wild-type Rep78 protein.

45. The method of claim 44, wherein said wild-type Rep78 protein is contacted with the same at least one DNA sequence obtained from one or more of a papillomavirus, an AAV, a HIV or an oncogene as contacted with said mutant for a period of time to allow binding of said mutant to said DNA; and compare the binding of said mutant with the binding of said wild-type Rep78 protein.
